(19) United States
(12) Patent Application Publication
(10) Pub. No.: US 2024/0060962 A1
(43) Pub. Date: Feb. 22, 2024

(54) DEVICE FOR MODULAR CONTROL OF MICROENVIROMENT FOR CELL MIGRATION AND CULTURE ASSAY AND METHOD FOR ITS USE

(71) Applicant: Oregon State University, Corvallis, OR (US)

(72) Inventors: Bo Sun, Corvallis, OR (US); Pedram Esfahani, Corvallis, OR (US)

(73) Assignee: Oregon State University, Corvallis, OR (US)

(21) Appl. No.: 18/448,091

(22) Filed: Aug. 10, 2023

Related U.S. Application Data
(60) Provisional application No. 63/397,540, filed on Aug. 12, 2022.

(51) Int. Cl. G01N 33/50; C12M 3/00; C12M 1/12; C12M 1/34; C12N 5/09

(52) U.S. Cl. CPC G01N 33/5011; C12M 23/42; C12M 23/48; C12M 25/04; C12M 41/46; C12N 5/0693; C12N 2513/00; C12N 2533/30

(57) ABSTRACT

Disclosed herein are embodiments of cell culture assays for simultaneous study of chemical and mechanical cues. Also disclosed herein are extracellular matrices for use with the cell culture assays. In some examples, the extracellular matrices can have a textured fibrous structure. Also disclosed herein are embodiments of a modular apparatus for forming the cell culture assays and the extracellular matrices used with the cell culture assays. Methods for using the cell culture assays are also disclosed herein.

### CROSS REFERENCE TO RELATED APPLICATION

This application claims the benefit of U.S. Provisional Patent Application No. 63/397,540, filed on Aug. 12, 2022, which is incorporated herein by reference in its entirety.

### ACKNOWLEDGMENT OF GOVERNMENT SUPPORT

This invention was made with government support under PHY1844627 awarded by National Science Foundation, R35GM138179 awarded by National Institutes of Health, and W81XWH-20-1-0444 awarded by the Department of Defense. The government has certain rights in the invention.

### FIELD

The present disclosure relates to cellular assay inserts, extracellular matrices for use in cellular assay inserts, and apparatus and methods for making the same.

### BACKGROUND

Cellular dynamics in many physiological processes are jointly regulated by biochemical and biophysical factors. Cells may respond simultaneously to chemical cues, such as chemotaxis, and mechanical cues, such as contact guidance from their environment. Thus, analyzing the response of cells to both chemical stimuli and mechanical stimuli is helpful in predicting the likely in vitro response of various cellular cultures to chemical exposure.

There exists, therefore, a need for improved testing environments that facilitate the simultaneous study of biochemical and biophysical stimuli, and methods for efficiently making and adapting the same.

### SUMMARY

Disclosed herein are cell culture assays, apparatus for preparing cell culture assays, and methods for preparing and using the same. The disclosed cell culture assays, preparation apparatus, and methods can, for example, provide improved ability to simultaneously measure chemical and mechanical cellular responses, and improved ability to control the relative alignment of chemical and mechanical cellular signals. The preparation apparatus and methods also can be quickly adjusted to prepare custom cell culture assays for a variety of test conditions. As such, the devices and methods disclosed herein can, among other things, provide an improved ability to predict cell responses to chemical and physical signals through various readily customizable tests.

Certain examples concern a device. The device comprises a cell culture container with a container body having a first end portion, a second end portion, and a coverslip attached to the first end portion of the container body. The container body defines a first reservoir, a second reservoir, and an observation chamber. The observation chamber is positioned between the first reservoir and the coverslip and extending between and open to the first reservoir and the second reservoir. The device also comprises an extracellular matrix positioned within an extracellular matrix chamber and separating the first reservoir from the second reservoir.

Certain examples concern a method. The method comprises preparing a precursor solution comprising a biopolymer, adding the precursor solution to a culture container comprising a first reservoir, a second reservoir, and a chamber separating the first reservoir and the second reservoir, and forming a fluid pathway from the first reservoir to the second reservoir, retaining the precursor solution in the culture container to nucleate a plurality of biopolymer fibers, and forming an extracellular matrix comprising the plurality of biopolymer fibers in the chamber.

Certain examples concern a device prepared according to the previously described method. The device comprises a culture container having a first reservoir, a second reservoir, and a chamber separating the first reservoir and the second reservoir. The chamber forms a fluid pathway from the first reservoir to the second reservoir. The device also includes an extracellular matrix comprising a network of interconnected biopolymer fibers and a cell culture disposed within the network of interconnected biopolymer fibers.

Certain examples concern a system. The system comprises a modular assembly with an interchangeable culture assay insert for adjustable preparation of a cell culture assay. The modular assembly includes a rotary stage having a base portion, a motorized mount comprising a rotating shaft, a motor that drives the rotating shaft, and a blade attached to the rotating shaft, and an adjustable positioning arm extending between the motorized mount and the base portion and movable in a first direction to change a relative position of the motorized mount to the base portion. The modular assembly also includes an assay holder positioned below the motorized mount and configured to receive a modular culture insert. The modular culture insert comprises a first reservoir, a second reservoir, a chamber separating the first reservoir and the second reservoir, and an aperture opening into the chamber. The aperture is configured to receive the blade of the motorized mount and admit the blade into the chamber when the rotary stage is in an operational state, and when the modular culture insert is received by the assay holder.

Certain examples concern a method for simultaneous measurement of a mechanical stimulus and a chemical stimulus on cells. The method comprises preparing a cell culture assay having a first reservoir, a second reservoir, a chamber in fluid communication with the first reservoir and the second reservoir, and an extracellular matrix disposed in the chamber and comprising a biopolymer and a cell culture. The method also comprises adding a first solution with a first concentration of an active chemical to the first reservoir, adding a second solution to the second reservoir to form a chemical gradient between the first reservoir and the second reservoir across the extracellular matrix, and measuring a response of the cell culture to the chemical gradient between the first reservoir and the second reservoir.

Certain examples concern a device. The device comprises a cell culture container having a container body with a first end portion, a second end portion, and a coverslip attached to the first end portion of the container body. The device also comprises an extracellular matrix comprising a network of interconnected fibers and a cell culture disposed within the extracellular matrix. The container body defines a first reservoir, a second reservoir, and an observation chamber extending between the first reservoir and the second reservoir. The extracellular matrix is positioned within the observation chamber and separates the first reservoir from the second reservoir.

Certain examples concern a cell culture container. The cell culture container comprises a container body having a first end portion, an open second end portion, and a coverslip attached to the first end portion of the container body. The cell culture container also comprises a first reservoir positioned within the container body, a second reservoir positioned within the cell culture container and separated from the first reservoir by a wall, and an observation chamber positioned between the first reservoir and coverslip, and open to the first reservoir. An aperture extends from the observation chamber to the open second end portion and is configured to receive a rotatable blade assembly and admit the rotatable blade assembly into the observation chamber, and a channel extends from the aperture to the second reservoir. The observation chamber, the aperture, and the channel together form a fluid pathway between the first reservoir and the second reservoir.

Certain examples concern a cell culture container. The cell culture container comprises a container body having a first end portion, a second end portion, and a coverslip attached to the first end portion of the container body. The cell culture container also comprises a first end reservoir and a second end reservoir defined by the cell culture container and open along the second end portion of the container body, a first observation chamber positioned between the first end reservoir and the coverslip, and open to the first end reservoir, a second observation chamber positioned between the second end reservoir and the coverslip, and open to the second end reservoir, an inner wall having a first end portion and a second end portion and disposed within the container body, and an inner reservoir defined by the inner wall. A first aperture extends through the inner wall and opens into the first observation chamber. A second aperture extends through the inner wall and opens into the second observation chamber. A first channel extends from the first aperture to the inner reservoir, and a second channel extends from the second aperture to the inner reservoir. The first observation chamber, the first aperture, and the first channel define a fluid pathway between the first end reservoir and the inner reservoir, and the second observation chamber, the second aperture, and the second channel define a fluid pathway between the second end reservoir and the inner reservoir.

The various innovations of this disclosure can be used in combination or separately. This summary is provided to introduce a selection of concepts in a simplified form that is further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

The patent or application file contains at least drawing executed in color. Copies of this patent or patent application publication with color drawing(s) will be provided by the Office upon request and payment of the necessary fee.

FIG. 1 is a front view of one embodiment of a modular rotary stage for preparing cell culture assays according to the present invention.

FIG. 2 is an exploded view of the modular rotary stage of FIG. 1.

FIG. 3A is a perspective view of the blade of the rotary stage in FIG. 2.

FIG. 3B is a side view of the blade of the rotary stage in FIG. 2.

FIG. 3C is a front view of the blade of the rotary stage in FIG. 2.

FIG. 4 is a schematic illustration of one embodiment of a vertical control of the rotary stage relative to an assay holder and culture container according to the present invention.

FIG. 5 shows an electronic control module for use with a rotary stage, according to one embodiment of the present invention.

FIG. 6A is a perspective view of one embodiment of a cell culture container.

FIG. 6B is a cutaway view of the cell culture container of FIG. 6A.

FIG. 7 is an exploded view of the cell culture container of FIG. 6.

FIG. 8 is a perspective view of another embodiment of a cell culture container according to the present invention.

FIG. 9 is a schematic illustration of one embodiment of a cell culture container comprising an extracellular matrix that forms a chemical pathway between an inner reservoir and an outer reservoir.

FIG. 10A are plan views showing the alignment of matrix fibers relative to each other in an extracellular matrix according to one embodiment of the present invention.

FIG. 10B shows the orientation of the fibers within the extracellular matrix.

FIG. 11 is a plot of the coherence values of the fibers of the extracellular matrix of FIGS. 10A and 10B, as a function of distance from the center of the observation chamber.

FIGS. 12A-12C shows the mechanochemical response of a cell culture in the extracellular matrix of FIG. 10.

FIG. 12A shows the position of individual cells of a cell culture in the extracellular matrix of FIG. 10 as a function of time.

FIG. 12B is a plot of the radial displacement of cells of a cell culture in the extracellular matrix of FIG. 10 as a function of time.

FIG. 12C is a plot of the circularity of the cells of the cell culture in the extracellular matrix of FIG. 10 as a function of aspect ratio.

FIGS. 13A-13B shows an extracellular matrix according to another embodiment of the present invention.

FIG. 13A shows the alignment of the matrix fibers relative to each other in an extracellular matrix.

FIG. 13B shows the orientation of the fibers within the extracellular matrix.

FIG. 14 is a plot of the coherence values of the fibers of the extracellular matrix of FIGS. 13A and 13B as a function of distance from the center of the observation chamber.

FIGS. 15A-15C show the mechanochemical response of a cell culture in the extracellular matrix of FIG. 13.

FIG. 15A shows the position of individual cells of a cell culture in the extracellular matrix of FIG. 13 as a function of time.

FIG. 15B is a plot of the radial displacement of the cells of a cell culture in the extracellular matrix of FIG. 13 as a function of time.

FIG. 15C is a plot of the circularity of the cells of the cell culture in the extracellular matrix of FIG. 13 as a function of aspect ratio.

FIG. 16 is a plot of the relative intensity of an active chemical within an extracellular matrix according to one embodiment of the present invention as a function of distance from the center of the observation chamber over time (or as a function of time).

FIG. 17A is a micrograph showing extracellular matrix fiber orientation as a function of varying agitation speed (64 RPM) and duration (2 minutes).

FIG. 17B is a micrograph showing extracellular matrix fiber orientation as a function of varying agitation speed (128 RPM) and duration (2 minutes).

FIG. 17C is a micrograph showing extracellular matrix fiber orientation as a function of varying agitation speed (128 RPM) and duration (4 minutes).

FIG. 18 is a micrograph showing extracellular matrix fiber density as a function of varying concentration (2-5 mg/mL) and polymerization temperature (25° C. and 37° C.).

FIG. 19 is a micrograph showing an extracellular matrix containing a multicellular construct, according to one example.

### DETAILED DESCRIPTION

### I. OVERVIEW OF TERMS

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise.

The term “includes” means “comprises.”

The term “coupled” generally means physically, mechanically, chemically, magnetically, and/or electrically coupled or linked and does not exclude the presence of intermediate elements between the coupled or associated items absent specific contrary language.

As used herein, the terms “axial” and “axially” refer to directions and orientations that extend substantially parallel to a centerline of a device having a substantially circular or rounded cross-section.

The terms “radial” and “radially” refer to directions and orientations that extend substantially perpendicular to the centerline of the device.

The terms “circumferential” and “circumferentially” refer to directions and orientations that extend arcuately about the centerline of the device.

### II. INTRODUCTION

Many physiological processes involve directed mechanochemical cues that regulate the motility, polarization and morphogenesis of cells, such as during wound healing, immune response, and cancer metastasis. It is therefore advantageous to incorporate these extracellular signals when employing in vitro, cell-based assays in applications such as drug screening and tissue regeneration. Disclosed herein is a modular platform to study cellular dynamics in a three-dimensional extracellular matrix where directed chemical and mechanical cues, in the forms of chemotaxis and contact guidance, are fully controlled in the cellular microenvironment.

Chemotaxis is one of the most common types of directed chemical cues and refers to a process where a cell follows a gradient of chemoattractants and/or chemorepellents to coordinate with other cells in the functions of multicellular organisms. Contact guidance, on the other hand, utilizes a substrate or tissue topography to direct cells through mechanosensing pathways. Contact guidance strongly modulates the morphology and motility of cells, as has been reported for many cell types.

Although the effects of chemotaxis and contact guidance have been well studied separately, accurate representation of physiological conditions is improved by simultaneous presence and control of mechanochemical cues (that is, both topographical cues and chemical cues). One salient example is cancer metastasis, where the gradient of various growth factors drives chemotaxis, which facilitates cancer cell dissemination. Concurrently, cells move through vast tissue space filled by fibrous extracellular matrix, and the alignment of extracellular matrix fibers generates contact guidance. As a result, extracellular matrix microstructure significantly correlates with tumor prognosis.

While it is desirable to program complex mechanochemical cues in cell-based assays, a reliable and user-friendly method has not been commercially available. To incorporate biochemical and biophysical factors in the microenvironment of the cells, techniques have been developed to pattern the microstructure of three-dimensional extracellular matrices. These techniques are packaged to a portable and modular platform: MC3A (Modular Control of Microenvironment for Cell Migration and Culture Assay). MC3A simultaneously controls mechanochemical factors for three-dimensional cultured cells, with a form factor compatible with standard microscopy for live or fixed cell imaging.

Disclosed herein are examples of portable modular platforms for patterning the microstructure of three-dimensional extracellular matrices. Generally, the disclosed modular platforms comprise a modular rotary stage, an electronic control module, and culture inserts. As will be described in greater detail below, the modular platforms are used to form textured, three-dimensional extracellular matrices for cellular and other assays.

### III. EMBODIMENTS OF THE DISCLOSED TECHNOLOGY

### A. Rotary Stage

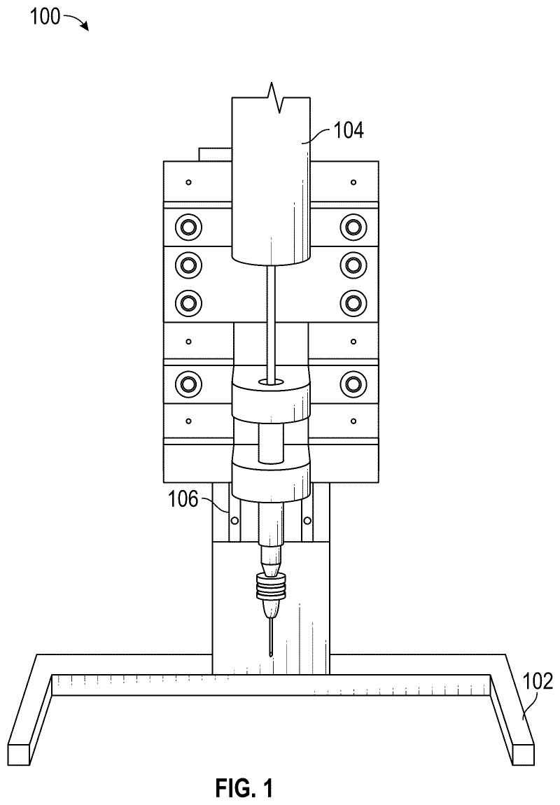

FIG. 1 depicts one embodiment of a disclosed example rotary stage 100 for modular platforms disclosed herein. As shown in FIG. 1, the rotary stage 100 comprises a base portion 102, a motorized mount 104, and a positioning arm 106. Positioning arm 106 extends between the base portion 102 and the motorized mount 104 and is configured to adjustably positioning the motorized mount 104 relative to the base portion 102 in one or more orthogonal axes (i.e., the x-axis, the y-axis, or the z-axis).

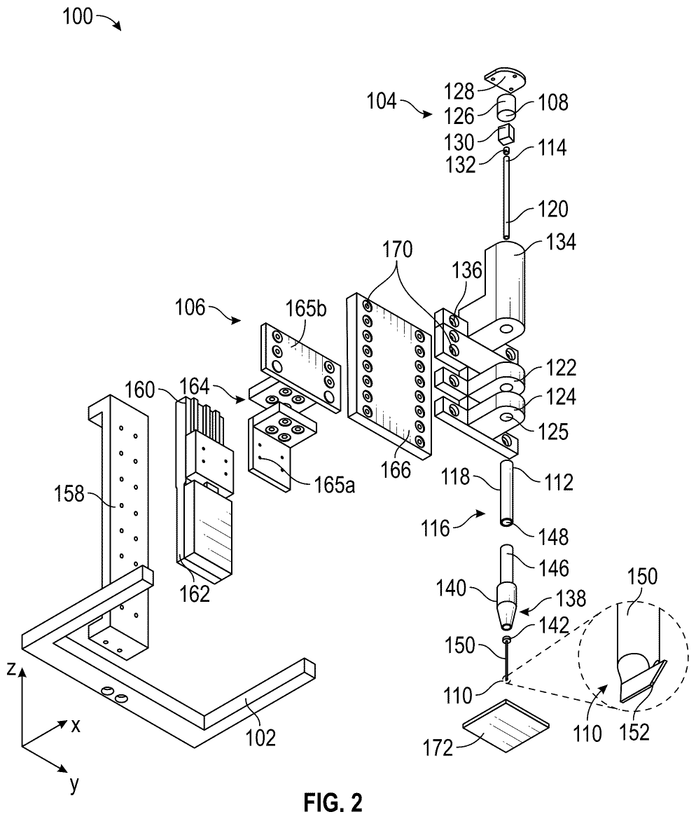

The components of the illustrated embodiment of the rotary stage 100 are illustrated in greater detail in FIG. 2. Motorized mount 104 comprises a motor assembly 108 coupled to a blade head 110 via a rotating shaft 112. Rotating shaft 112 can extend along a first orthogonal axis (labeled “Z” in FIG. 2) of the rotary stage between the motor assembly 108 (at a first end portion 114 of the rotating shaft 112) and the blade head 110 (at a second end portion 116 of the rotating shaft 112). Rotating shaft 112 can comprise one or more rigid components, such as a metal rod 118, and optionally one or more pliant components, such as a rubber rod 120, that absorb vibrations caused by the motor assembly 108, thereby minimizing unintended displacement of the blade head 110.

The motorized mount 104 may also include one or more aligners. For example, as shown in FIG. 2, the motorized mount includes a first aligner 122 and a second aligner 124. The aligners 122, 124 each have an aperture 125 extending therethrough, which receives the rotating shaft 112, such that the first end portion 114 and the second end portion 116 of the rotating shaft 112 are positioned at opposite sides of the aligners 122, 124. The aligners 122, 124 may be independently adjustable along a second orthogonal axis (labeled “Y” in FIG. 2) perpendicular to the first orthogonal axis Z so that any angle between the rotating shaft 112 and the first orthogonal axis Z can be controlled by adjusting the position of the aligners 122, 124.

With continued reference to FIG. 2, the motor assembly 108 can include a DC step motor 126 and a motor box cap 128. The motor assembly 108 can also include a gear box 130 positioned between the step motor 126 and the rotating shaft 112. In some examples, the rotating shaft 112 can be connected to the gear box by an attaching rod 132. While a motorized mount 104 using a stepper motor is shown in FIG. 2, a person of ordinary skill in the art will understand that other types of motors may also be suitable for use in the motorized mount, such as AC motors and/or servomotors. The motors may be manually or automatically controlled.

As shown in FIG. 2, the motor assembly 108, the attaching rod 132, and portions of the rotating shaft 112 can be contained in a motor compartment 134. The motor compartment 134 encloses and secures the various components of the motor assembly 108 and the fixtures connecting the motor assembly 108 to the rotating shaft 112.

The second end portion 116 of the rotating shaft 112 is coupled to a blade assembly 138. In some examples, the blade assembly 138 is statically mounted to the rotating shaft 112, such that when the motor assembly 108 turns, the rotating shaft 112 and the blade assembly 138 rotate together. As shown in FIG. 2, the blade assembly 138 comprises a rotatable fixture 140, a blade mount 142, and the blade head 110. The rotatable fixture 140 can be coupled to the rotating shaft 112, for example, by a rod 146 that slots into a corresponding bore 148 in the rotating shaft, as shown in the illustrated example. In turn, the blade mount 142 can be coupled to the rotatable fixture 140, for example, by a magnet disposed in the blade mount that attaches the blade mount to the rotatable fixture. While the example illustrated in FIG. 2 shows the rotatable fixture 140 coupled to the rotating shaft 112 by a rod received by a corresponding bore, and the blade mount 142 attached to the rotatable fixture 140 by a magnet, a person of ordinary skill in the art will understand that a variety of other mechanisms can be used to secure these components to one another, such as corresponding male and female screw threads, press friction fittings, clamps, adhesives, or locking notch and groove configurations.

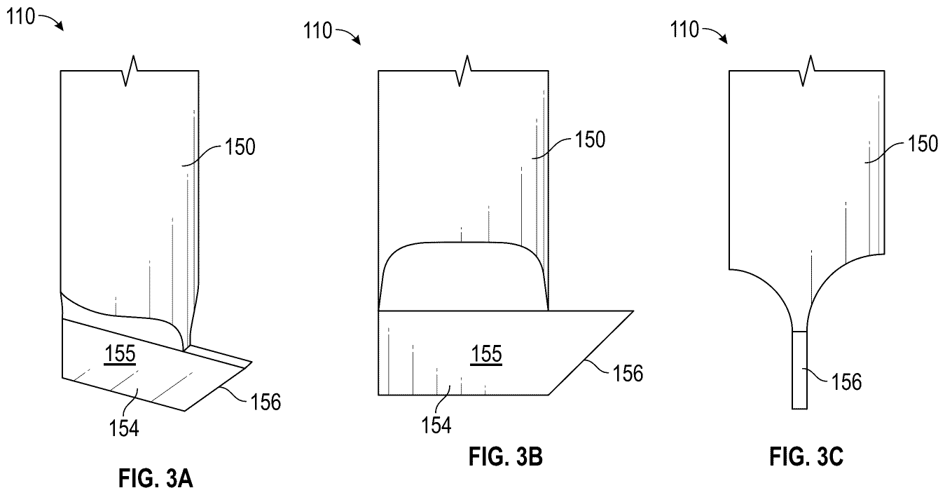

The blade assembly 138 can also comprise a rod 150 (or spindle 150) coupled to the blade mount 142 at one end and coupled to the blade head 110 at the other end. The blade head 110 is illustrated in greater detail in FIGS. 3A through 3C and comprises a rectangular body 154 attached to the end portion of the rod 150, and a wedge-shaped protrusion 156 extending laterally outwards from the rectangular body 154 and past the rod 150.

Because the blade assembly 138 is coupled to the rotating shaft 112, when the motor assembly 108 drives the rotating shaft 112, the blade assembly 138 rotates along with the rotating shaft 112. When the rotating blade head 152 is immersed in a liquid medium (such as a biopolymer solution), the rotational motion of the blade head 152 causes a flow in the liquid medium that depends on the rotational velocity and the shape of the blade head 152, as will be discussed in greater detail below. Other geometries may be desirable for the blade head 152 in other processes, depending on the desired flow behavior of the liquid medium. In some specific examples, it may be particularly advantageous for the geometry of the blade head 152 to correspond to the geometry of the chamber containing the liquid medium.

In some examples, the blade surface 155 can be further functionalized by a coating. Such coatings can include, but are not limited to, hydrophobic or hydrophilic coatings, depending on specific applications and the types of extracellular matrix polymer used. For example, hydrophobic coatings can be added to prevent a biopolymer precursor solution, such as those described in greater detail below, from wetting the blade surface 155 and nucleating biopolymer fibers. Hydrophobic coatings can, in some examples, include polydimethylsiloxane, fluorinated silanes, or any combination thereof, and may be applied through dip coating or vapor deposition. In other examples, it may be desirable to encourage fiber nucleation, and so a hydrophilic coating could be added to encourage wetting and nucleation. Hydrophilic coatings can, in some examples, comprise polyacrylic acid, polyethylene oxide, or any combination thereof, which can be applied by dip coating or vapor deposition. In other examples, the blade surface 155 can be rendered temporarily hydrophilic by oxygen plasma or corona treatments.

Returning to FIG. 2, the aligners 122, 124, and the motor compartment 134 are coupled to one another to form an alignment stage 136 of the motorized mount 104. In this way, the motor assembly 108, the aligners 122, 124, the rotating shaft 112 extending through the aligners 122, 124, and the blade assembly 138 can be positioned relative to the base portion 102 of the rotary stage 100, while held in a fixed position relative to each other.

The rotary stage 100 can also include a positioning arm 106. As shown in FIG. 1, the positioning arm 106 extends between the motorized mount 104 and the base portion 102 of the rotary stage 100. The positioning arm 106 can be configured to allow positioning of the motorized mount 104 in three dimensions, relative to the base portion 102 of the rotary stage. That is, the components of the positioning arm 106 can be movable in the first orthogonal axis Z, the second orthogonal axis Y, and a third orthogonal axis (labeled “X” in FIG. 2) perpendicular to the first orthogonal axis Z and the second orthogonal axis Y.

As best shown in FIG. 2, the positioning arm 106 can comprise a first vertical member 158 and a second vertical member 160 coupled to the first vertical member 158. In some examples, the second vertical member 160 can further include a first motorized jack 162 that allows the second vertical member 160 to be moved relative to the first vertical member 158 along the first orthogonal axis Z.

The positioning arm 106 can also include an angled mount 164 and a mounting plate 166. As shown in FIG. 2, the angled mount 164 can further comprise a first L-shaped bracket 165a and a second L-shaped bracket 165b. The angled mount 164 extends from the second vertical member 160 to the mounting plate 166, coupling the mounting plate 166 to the second vertical member 160 and spacing the mounting plate 166 in the second orthogonal direction. That is, positioning the mounting plate 166 forward of the second vertical member 160, as shown in FIG. 2.

The mounting plate 166 can extend from the angled mount 164, and more particularly extend from the second L-shaped bracket 165b to the alignment stage 136 of the motorized mount 104. The alignment stage 136 of the motorized mount 104 also can be fixedly coupled to the mounting plate 166 of the motorized mount 104 and thereby operatively connected to the positioning arm 106.

While the examples described above use a motorized jack 162 to actuate the positioning arm, other translocation mechanisms, including multi-axis motorized or manual stages, can be used to position the spinning blade head 152.

In some examples, such as that illustrated in FIGS. 1 and 2, the components of the positioning arm 106 can be fastened to each other and to the base portion 102 and the motorized mount 104 by a plurality of bolts or screws extending through a plurality of apertures 170. Other securing mechanisms can be used to secure the components of the positioning arm 106 to one another and to the base portion 102 and the motorized mount 104, such as a plurality of pins extending into a plurality of sockets, or a plurality of tongue-and-groove fixtures. A person of ordinary skill in the art will also appreciate that certain components of the positioning arm 106 illustrated as being statically fixed relative to one another can be integrally formed, such that no further securing mechanism between those components is necessary.

As previously discussed, and as illustrated in FIG. 1, the base portion 102 of the rotary stage 100 generally supports the other components of the rotary stage, such as the motorized mount and the positioning arm. The base portion 102 can also define a workspace that receives a culture container (such as culture container 200, discussed in greater detail below) on an assay holder 172, as illustrated in FIG. 2, to position the culture container relative to the blade assembly 138.

Particularly, in some examples, the rotary stage 100 may include one or more motorized jacks 162 configured to move the assay holder 172 (and any culture container used with assay holder 172) along a second orthogonal axis Y perpendicular to the first orthogonal axis Z and a third orthogonal axis X perpendicular to both the first orthogonal axis Z and the second orthogonal axis Y. In this way, the assay holder 172 can be positioned in a two-dimensional plane (i.e., the x-y plane), while the first motorized jack 162 of the positioning arm 106 moves the blade assembly in the first orthogonal direction (i.e., along the z-axis), allowing for full control of the relative positioning between the culture container and the blade assembly in three dimensions.

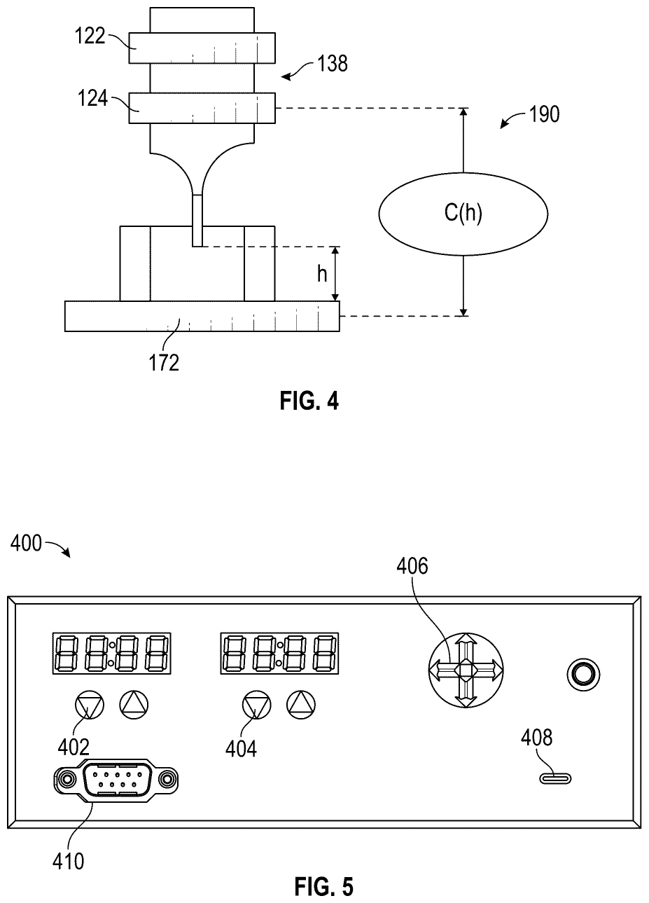

It may be advantageous to keep the blade head 152 at a certain height above the bottom of the cell culture container. Fine adjustment capability to the height of blade head 152 also allows additional control over the resulting tissue microstructure. Accordingly, it may also be advantageous for the rotary stage 100 to include mechanisms for monitoring the height of the blade assembly 138 relative to the base portion 102 (and thus to the culture container). In some examples, the relative positions of the base portion 102 and the blade assembly 138 are measured by capacitance, such as by using a capacitance detector 190 as illustrated in FIG. 4. In such examples, the spacing between the base portion 102 and the blade assembly 138 can be measured by monitoring the capacitance (for example, by a capacitance sensor) between two metal parts, such as the assay holder 172 and the first or second aligners 122, 124. As the parts move further apart, the capacitance between them decreases measurably. As the parts move closer together, the capacitance between them increases measurably. Thus, by observing the capacitance measured by the capacitance detector 190, the relative position of the assay holder 172 and the second aligner can be measured. It will be understood, however, that other methods, such as laser reflection, interferometry, or a touch pressure sensor could also be used to measure the relative positioning between the base portion 102 and the blade assembly 138, and to provide position data to the control module as will be described in greater detail below.

The embodiments previously discussed describe controlling the relative position of the assay holder 172 (and any culture container used with the assay holder 172) and the blade assembly 138 relative to each other by actuating the positioning arm 106 in one orthogonal direction and actuating the assay holder 172 in two orthogonal directions. However, a person or ordinary skill in the art will understand that, in some implementations, the positioning arm 106 may include one or more additional motorized jacks 162 to allow the motorized mount 104 to be positioned in two or three orthogonal directions. Advantageously, the modular design of the positioning arm allows such additional motorized jacks 162 to be positioned between any or all of the components of the positioning arm previously mentioned, including at least the first vertical member 158, the second vertical member 160, the first and second L-shaped brackets 165a, 165b of the angled mount 164, and the mounting plate 166. These additional motorized jacks can be in lieu of or in addition to the motorized jack 162 of the second vertical member 160, and any actuation mechanism for the assay holder 172.

Likewise, the first motorized jack 162 can, in some implementations, be positioned elsewhere on the positioning arm 106. For example, the first motorized jack 162 can be positioned between or on any or all of the components of the positioning arm previously mentioned, including at least the first vertical member 158, the second vertical member 160, the first and second L-shaped brackets 165a, 165b of the angled mount 164, and the mounting plate 166.

The rotary stage 100, described previously and illustrated in FIGS. 1 and 2, comprises a separate base portion 102, motorized mount 104, and positioning arm 106, that are assembled and affixed together, for example, by bolts, screws, sockets, pins, and other securing methods as previously described. Any of the base portion 102, the motorized mount 104, and the positioning arm 106 also can be integrally formed with one another, which may offer certain advantages, such as reducing any tolerance mismatch and/or movement between the components.

The portions of the rotary stage 100, such as the base portion 102, the motorized mount 104, the positioning arm, and the components thereof can be made of any durable solid material such as iron, stainless steel, aluminum, ceramics, and plastic. The embodiment illustrated by FIG. 2 was made using polylactic acid (“PLA+”).

### B. Culture Container

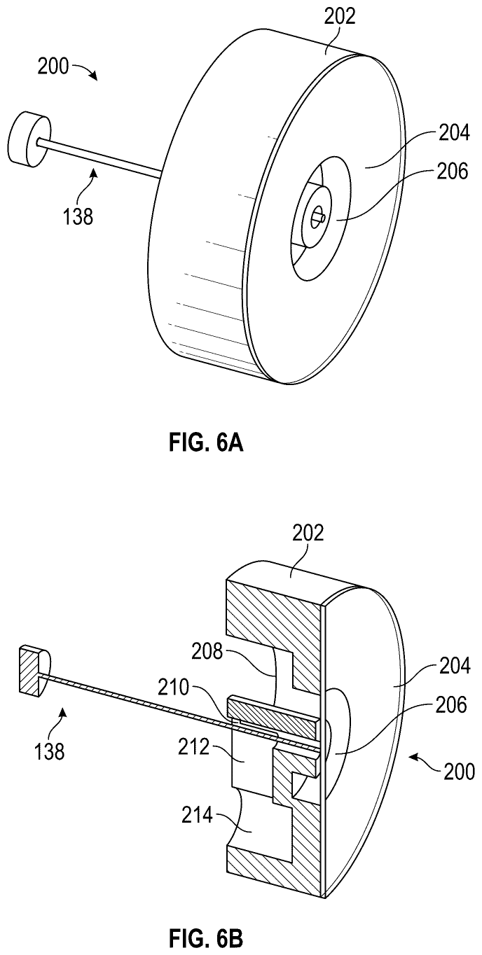

The modular platforms for patterning the microstructure of three-dimensional extracellular matrices disclosed herein generally also include a modular culture container for performing physiochemical assays. One example of a disclosed culture container 200 is illustrated in FIG. 6A. The culture container 200 includes a container body 202 and a coverslip 204 coupled to the container body 202. Container body 202 comprises an observation chamber 206 that is open to the coverslip 204.

FIG. 6B shows a cutaway view of the culture container 200 illustrated in FIG. 6A. The observation chamber 206 is open to (and in fluid communication with) a first reservoir 208 and to an aperture 210 defined by the container body 202. The observation chamber 206 can, in some examples, be positioned between the first reservoir 208 and the coverslip 204. The aperture 210 is configured to receive an agitation device, such as the blade assembly 138 described above and illustrated in FIGS. 1 through 3A. The container body 202 also defines a channel 212 that extends radially from the aperture 210 to a second reservoir 214. Channel 212 allows fluid communication between the first reservoir 208 and the second reservoir 214 through the aperture 210 and the channel 212.

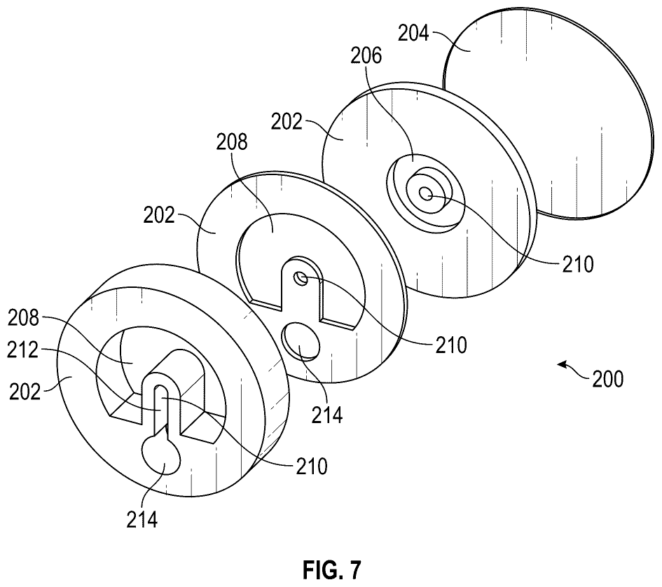

As shown in FIGS. 7A-7B, the observation chamber 206 is visible through the coverslip 204. This advantageously facilitates observation (e.g., by microscopy) of the behavior of extracellular matrices and cell cultures when they are located in the observation chamber 206, as will be discussed in greater detail later.

The respective positions of the first reservoir 208, the aperture 210, the channel 212, and the second reservoir 214 are illustrated in greater detail in FIG. 7, which depicts an exploded view of the culture container 200. As shown in the illustrated example, the aperture 210 can extend axially through the culture container 200. The first reservoir 208 can be spaced radially outwards from the aperture 210 and extend along a partial circumference of the container body 202. The second reservoir 214 can be radially spaced apart from the aperture 210 and circumferentially separated from the first reservoir 208, connected to the first reservoir only through the channel 212 that extends between the second reservoir 214 and the aperture 210.

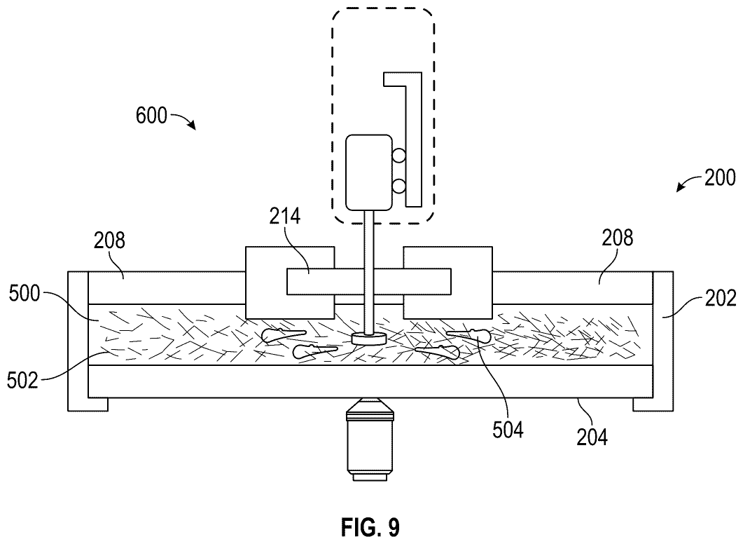

A chemical gradient can be formed between the first reservoir 208 and the second reservoir 214. For example, a first solution comprising a chemoattractant and/or a chemorepellent with a first concentration can be placed in the first reservoir 208, and a second solution with a second concentration can be placed in the second reservoir 214. When the first concentration and the second concentration are different, for example by at least 10% by volume concentration, or by a difference of 20% by volume in one particular example, a chemical gradient is formed between the first reservoir 208 and the second reservoir 214, as schematically represented in FIG. 9.

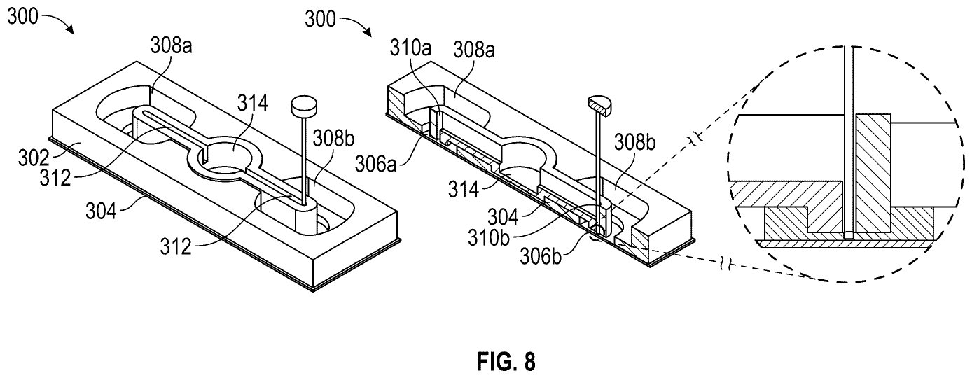

FIG. 8 illustrates another example modular culture container 300, with the capacity to run two physiochemical assays simultaneously. The modular culture container 300 is designed and functions in the same or substantially the same way previously discussed in relation to the modular culture container 200 and illustrated in FIGS. 6A-7, except for the differences described below.

As shown in FIG. 8, the culture container 300 comprises a body 302 and a coverslip 304 coupled to the body 302. A first observation chamber 306a and a second observation chamber 306b are open to the coverslip 304 to allow a user to inspect the contents of the culture container 300 through the coverslip.

The body 302 also defines two outer reservoirs 308a, 308b, and two apertures 310a, 310b. A channel 312 extends from each of the apertures 310a, 310b to a central reservoir 314. The apertures 310a, 310b are open to the channels 312 and to the outer reservoirs 308a, 308b, so that the outer reservoirs 308a, 308b are in fluid communication with the central reservoir 314. As shown in FIG. 8, the outer reservoirs 308a, 308b are not in communication with each other, except for through their mutual connection to the central reservoir 314. In some examples, the central reservoir can include one or more dividers to separate the central reservoir 314 into two or more chambers.

In a way similar to that described above in relation to modular culture container 200, a chemical gradient can be formed between the center reservoir 314 and the outer reservoirs 308a, 308b. The modular culture container 300 allows physiochemical assays to be run side by side. For example, a first solution could be added to the outer reservoir 308a, and a second solution could be added to the outer reservoir 308b, allowing the cytochemical effect of different solutions on a cellular culture to be measured side by side, controlling for experimental variables apart from the differences in the solutions added to the outer reservoirs.

The culture containers 200, 300 described herein can be formed of any suitable material, and may be additively manufactured, injection molded, or otherwise machined to the desired geometric configurations. Suitable materials for the culture containers 200, 300 include those which are chemically inert with respect to any chemicals used to form the gradients described above, and those which are non-cytotoxic. Specific examples may be formed from polylactic acid (PLA) or polydimethylsiloxane (PDMS).

### C. Electronic Control Module

The modular platforms for patterning the microstructure of three-dimensional extracellular matrices disclosed herein generally also include an electronic control module. The electronic control module hosts electronic components that allow user control over the operation of the rotary stage 100. For example, the control module can be configured to allow the user to control the relative positions between motorized mount 104 (and particularly the blade assembly 138) and a culture container, such as culture containers 200 and 300. The electronic control module may also be configured to allow user input over other factors, such as the rotational speed of the blade assembly 138.

FIG. 5 illustrates one example electronic control module 400. The illustrated control module embodiment includes a height control input 402, a rotational speed input 404, a horizontal position input 406, and a machine control output port 410. The height control input 402, the rotational speed input 404, and the horizontal control input 406 can be configured for direct, manual input of operational parameters for the rotary stage 100 and can advantageously allow a user to make fine adjustments to the rotary stage 100 without using a software interface.

The control module 400 can also include a computer input port 408, such as a USB port as a computer interface such as LabVIEW. The computer input port 408 can receive inputs from a computerized control program in lieu of, or in addition to, manual inputs from the user. This allows programming the modular platform therefor having more control on varying parameters such as but not limited to speed profiles, complex position profiles, or profiles using multiple culture containers.

The control module 400 also includes a machine control output port 410 comprising a plurality of control terminals. The machine control output port can convey operational signals from the control module 400 to the motors of the rotary stage 100, allowing user control over variables such as the positioning of the blade assembly 138 and the rotational speed of the blade assembly 138. For example, as illustrated in FIG. 2, the motor assembly 108 can be electronically connected to and controlled by a first control terminal. Likewise, the aligners 122, 124 can be connected to a second control terminal, the assay holder 172 to a third control terminal, and the motorized jack 162 to a fourth control terminal. The various control terminals transmit electronic signals to the respective connected components of the rotary stage 100 and control the operational position and characteristics of the rotary stage 100.

In some examples, such as that conceptually illustrated in FIG. 5, the control module 400 can also include a capacitance monitor. The capacitance monitor can be configured to measure the relative position of the blade assembly 138 and the assay holder 172, and therefore also the relative positions of the blade head 152 and the culture container 200, 300. Because it may be advantageous in some examples to maintain the blade head 152 at a given distance from the bottom of the culture container 200, 300, the use of the capacitance monitor can allow for automatic optimization of the blade head position relative to the culture container 200, 300.

### D. Extracellular Matrix Assays

Also disclosed herein are examples of three-dimensional extracellular matrices that can be prepared using embodiments of the modular platforms disclosed herein. These example extracellular matrices can be combined with a culture container (such as culture containers 200 and 300 described above) to form an extracellular matrix assay.

Typically, the three-dimensional extracellular matrix comprises a fibrous structure that mimics living tissues for improved control over and ability to measure mechanochemical cues. The fibrous structure of the extracellular matrix provides mechanical and/or physical cues to culture cells retained in the extracellular matrix and provides a scaffolding network for movement and/or migration of culture cells through the extracellular matrix, such as in the case of cellular chemotaxis.

The extracellular matrices disclosed herein may comprise a fiber-forming biopolymer. In some examples disclosed herein, the biopolymer is collagen; however, a person of ordinary skill in the art will appreciate that other biopolymers including fibronectin, elastin, laminin, vitronectin, or any combination thereof may also be suitable for forming an extracellular matrix with an interconnected fibrous network.

In addition to the combinations of the biopolymers discussed above for forming fibrous or non-fibrous extracellular matrixes, any extracellular matrix may include one or more gelling agents or cross-linking agents. The gelling and/or cross-linking compounds can include, for example, PEG-diNHS, PEGSSDA, Lysyl oxidase, methacrylation agents, and tetramethylethylenediamine. Advantageously, the inclusion of a gelling agent or a cross-linking agent may allow a user to control the gelation temperature, the gelation speed, the porosity, and/or the physical properties of the extracellular matrix according to the needs of the specific cellular culture desired. The various biopolymers and cross-linking agents and their respective concentrations can be varied to control the physical properties of the resulting extracellular matrix, such as porosity, pore size, and Young's modulus. For example, by combining various biopolymers and cross-linking agents and their respective concentrations, an extracellular matrix can be formed with a Young's modulus ranging from 1 Pa to 10 MPa.

In some examples, the extracellular matrix may comprise one or more hydrogels, including poly(ethylene glycol), alginate, gelatin, silk fibroin, polyethylene glycol diacrylate, and hyaluronic acid, or a combination thereof. In some examples, the extracellular matrix can comprise Matrigel, a hydrogel comprising a combination of collagen IV, laminin, perlecan, and various growth factors. Hydrogels can be in lieu of or in addition to the biopolymers described above. That is, in some examples, the extracellular matrix may comprise the hydrogel alone, with the biopolymer omitted.

The extracellular matrices disclosed herein may be configured to receive and support a cell culture. In some examples, the extracellular matrix can be configured to receive and support a culture of MDA-MB-231 cells (human breast cancer cells). In other examples, the extracellular matrix can be configured to receive and support a culture of endothelial cells. In other examples, the extracellular matrix can be configured to receive and support a culture of immune cells (such as human T-cells). In other examples, the extracellular matrix can be configured to receive and support a culture of stem cells.

The extracellular matrices disclosed herein may also be configured to receive a multicellular construct. In contrast to a cell culture, which in general form may comprise a plurality of individual cells, a multicellular construct comprises a plurality of interconnected cells organized into a structure, such as a tumor spheroid or a tissue organoid.

The specific nature and behavior of the cell culture or multicellular construct to be developed in the extracellular matrix may impose requirements on the extracellular matrix. For example, some cell cultures or multicellular constructs may require a greater or lesser degree of porosity in the extracellular matrix, and the porosity of the extracellular matrix can influence the speed of cellular movement and/or cellular response to physical and/or chemical stimuli, for example, by chemotaxis, through the extracellular matrix. When the porosity is too low, for example, when the pore size is smaller than the cell nucleus, some cell cultures may be unable to travel through the extracellular matrix or may only travel very slowly through the extracellular matrix because the spacing between the fibers is too small. When the porosity is too high, there may be too much space between the individual fibers of the extracellular matrix, which may prevent the cells from crossing from one fiber to another. Because the size and motility of the cell culture will vary depending on the cells included, and because different studies may require different travel speeds, the desired porosity of the extracellular matrix may vary depending on the combination of cell culture and stimulants, as well as the measured response. This porosity can be controlled by adjusting the type, amount, and concentration of the gelling and/or cross-linking agents added to the extracellular matrix. In some examples, the extracellular matrix can have a pore size ranging from 1 nm to 100 m.

Some cell cultures or multicellular constructs may need to be kept above or below certain thresholds of pH and temperature. For instance, if the extracellular matrix becomes too acidic or too basic, it may kill or damage the cell culture or multicellular construct. This can be controlled by the addition of acidic or basic compounds to adjust the pH of the extracellular matrix. Likewise, if the matrix requires a gelation temperature that is too high or too low, the temperature exposure may kill or damage the cell culture or multicellular construct during the extracellular matrix formation. This can be controlled by the addition of cross-linking agents and/or other compounds that elevate or depress the temperature at which the extracellular matrix begins to nucleate from the precursor solution.

A person of ordinary skill in the art will therefore appreciate that the addition of various reagents and components as described above can allow the extracellular matrix to be tailored to desired cellular culture or multicellular construct characteristics.

In specific examples described herein, the extracellular matrix comprises a fibrous three-dimensional structure formed from type-I collagen fibers. The extracellular matrix supports a culture of MDA-MB-231 human breast cancer cells. In other examples described herein, the extracellular matrix supports multicellular constructs, including tumor spheroids, issue organoids, and other compact multicellular constructs.

The extracellular matrix can be formed in a culture container, such as the culture containers 200 and 300 described with reference to FIGS. 6-8, to form an extracellular matrix assay. The extracellular matrix forms a cell-permeable connection between separate reservoirs (such as the first reservoir 208 and the second reservoir 214 of culture container 200). Thus, the cell culture can be exposed to a chemical gradient between the separate reservoirs, such as those described above.

FIG. 9 illustrates an example extracellular matrix assay 600, comprising an extracellular matrix (ECM) 500 and a culture container 200. The extracellular matrix 500 is contained within the container body 202, is positioned between the coverslip 204 and the reservoirs 208, 214, and comprises a plurality of interconnected fibers 502 that form a fibrous scaffold or network. A culture of cells 504 is contained within the extracellular matrix 500. Cells 504 can respond to various mechanochemical stimuli, depending on the type of assay being conducted. As an example, the first reservoir 208 and the second reservoir 214 are separated by the extracellular matrix 500. This allows solutions of different concentrations to be placed in the first reservoir 208 and second reservoir 214, as previously described. Because a chemical gradient is formed between the first reservoir 208 and the second reservoir 214, cells 504 experience both the chemical stimulus of the chemical gradient and the mechanical stimulus of the extracellular matrix 500. This causes simultaneous chemotaxis and contact guidance of the cells 504.

If, for example, the first reservoir 208 is left chemically neutral (i.e., serum-free), and the second reservoir 214 is filled with a solution containing compounds which are repellant to the cells 504, such as bone morphogenetic proteins (BMP) and 8CPT-cAMP, negative chemotaxis will cause the cells 504 to travel radially outwards towards the first reservoir 208 along the matrix of interconnected fibers 502. However, if the first reservoir 208 is left chemically neutral, and the second reservoir 214 is filled with a solution containing compounds which attract the cells 504, including serums such as fetal bovine serum (“FBS”) or calf bovine serum (“CBS”), glucose, cyclic adenosine monophosphate (“cAMP”), or transforming growth factor β (“TGF-β”) chemotaxis will cause the cells 504 to travel radially inwards towards the second reservoir 214 along the matrix of interconnected fibers 502.

The interconnected fibers 502 of the extracellular matrix 500 may, in various examples, have varying degrees of fiber alignment. The degree of fiber alignment can depend on a variety of factors, such as the biopolymer used, the temperature and time of the gelation process, and any agitation and/or stirring, such as that which can be provided by the blade head 152, as described above.

The degree of alignment between fibers on a local level (that is, the alignment of individual fibers relative to neighboring fibers) can be expressed as a coherence value, “c”. When the orientation of individual fiber relative to neighboring fibers is evenly distributed in all 360 degrees, c is 0. When the fibers are parallel to neighboring fibers (that is, when all fibers have the same orientation), c is 1.

The coherence value, c, can be visually measured using image analysis software. For example, micrographs can be prepared for an extracellular matrix 500, showing the microstructure of the extracellular matrix 500, such as the fibers 502. The micrographs can be analyzed by software, such as an ImageJ plugin, OrientationJ, that is capable of quantifying the overall orientation of the fibers 502 of a given window around the desired coordinate, and the local values of c, based on the image. More specifically, the software computes the orientational order of an image based on its gradient matrix. To determine the coherence, c, the software first identifies a principal direction of fibers orientation from an image of the extracellular matrix 500. With the principal direction identified, the software then evaluates the local orientation of the fiber matrix relative to the identified principal direction. Because c is a measurement of local fiber coherence, the value of c can vary at different locations within a single extracellular matrix.

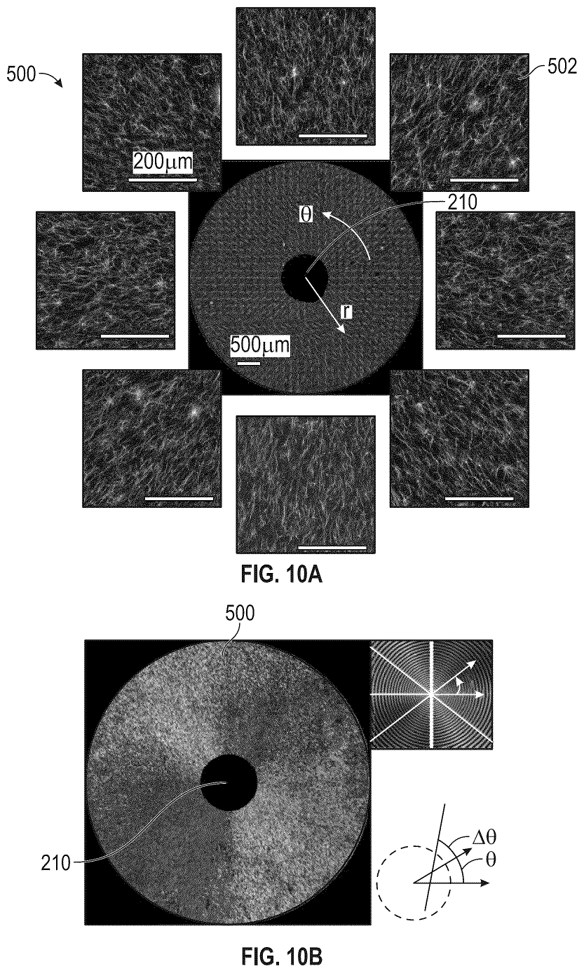

In some implementations, biopolymer solution gelation may take place without rotational agitation by the blade assembly 138 of the motorized mount 104, or with the blade assembly 138 rotating at a low speed (in some examples, rotating at a speed of greater than 0 RPM to 20 RPM or less), the biopolymer will gel to form a radially aligned fibrous extracellular matrix 500, as illustrated in FIGS. 10A and 10B. The individual fibers 502 of the radially aligned extracellular matrix 500 extend from the first portion of the culture container, the aperture 210, to an external periphery of the extracellular matrix, as shown.

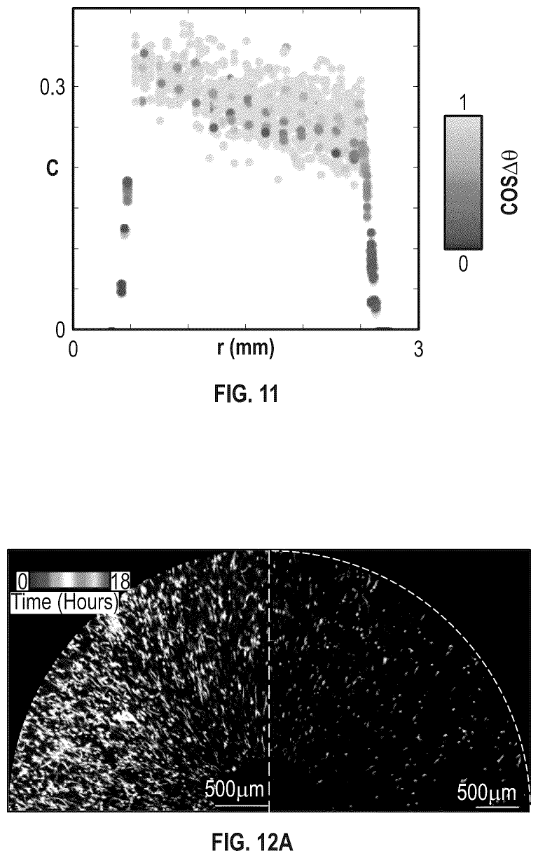

The coherency value c of the fibers of the extracellular matrix can vary depending on the location within the extracellular matrix. In one example, shown in FIG. 11, the extracellular matrix comprised of a fiber network has an average coherency of approximately 0.30 near the second reservoir 214. As the distance from the second aperture 210 increased, the coherence value c of the fibers gradually decreased to a value of approximately 0.25.

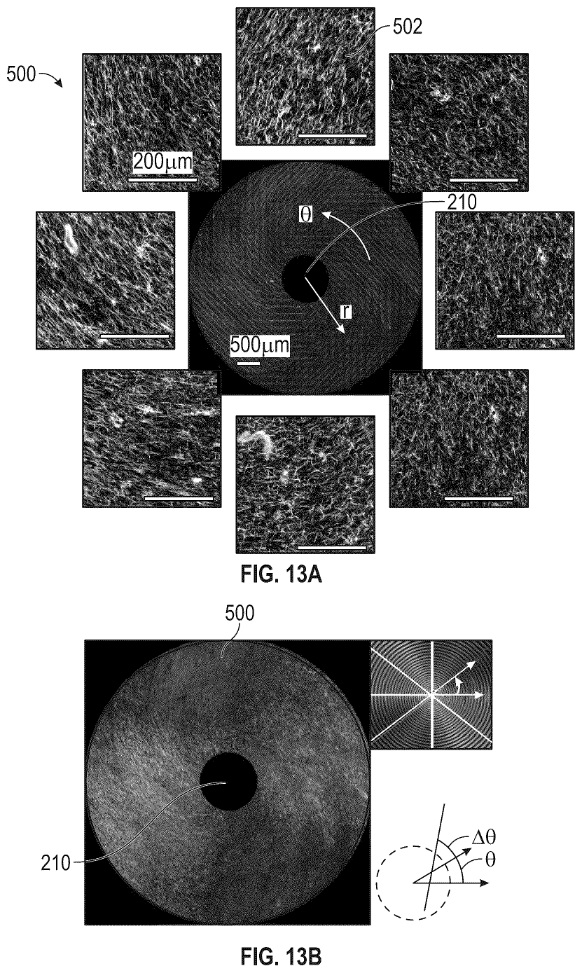

In other implementations, the biopolymer may be stirred or rotationally agitated by the blade head 152 of the motorized mount 104, with the blade head 152 rotating at high speed (such as greater than 20 RPM to at least 128 RPM). In such implementations, the induced flow of the biopolymer solution causes nucleation of fibers 502 that extend in the circumferential as well as the radial directions. This forms an extracellular matrix with a circumferentially aligned fibrous structure, such as that shown in FIGS. 13A and 13B. In a circumferentially aligned extracellular matrix, the individual fibers 502 extend radially from the first portion of the culture container, such as the second reservoir 214, to an external periphery of the extracellular matrix, as shown.

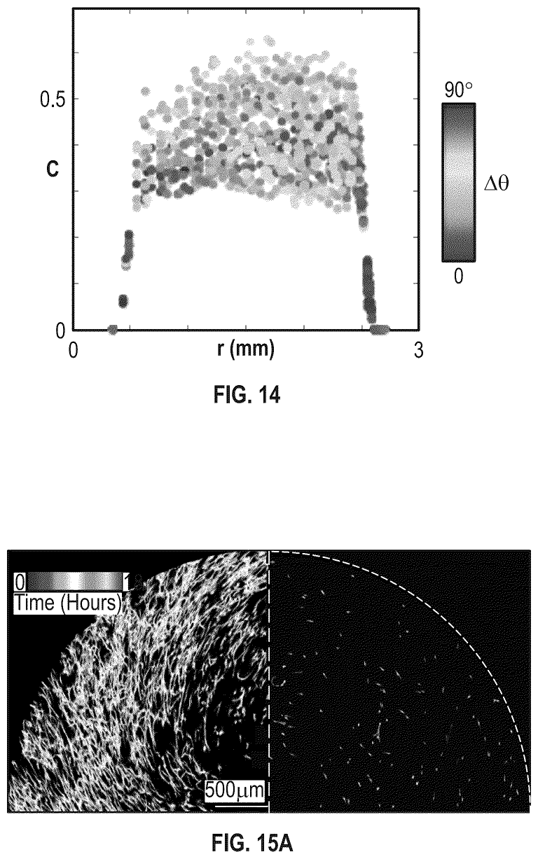

Again, the coherency value c of the fibers of the circumferentially aligned extracellular matrix can vary depending on the location within the extracellular matrix. In one example, shown in FIG. 14, the extracellular matrix comprised a fiber network with an average coherency of approximately 0.40 near the second reservoir 214 and along the external periphery of the extracellular matrix 500. As the distance to the radial midpoint (i.e., the circle equidistant from the external periphery of the extracellular matrix 500 and the second reservoir 214) decreased, the coherency of the fibers 502 increased to an average of approximately 0.5.

While the extracellular matrices described above have a generally fibrous structure, with the biopolymer forming a matrix or scaffold of interconnected strands, a person of ordinary skill will appreciate that the extracellular matrix may have a different general structure. For example, in some circumstances, it may not be necessary to measure a response to mechanical stimuli, such as the presence of a fibrous network structure. In such examples, the extracellular matrix can be formed of one or more biogels, including Matrigel, poly(ethylene glycol), alginate, gelatin, silk fibroin, polyethylene glycol diacrylate, hyaluronic acid, or any combination thereof to allow for isolation of responses of the cell culture to chemical responses.

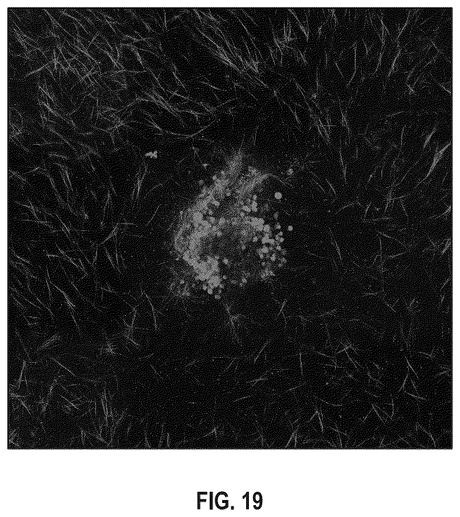

An alternative extracellular matrix 700 comprising an interconnected network of biopolymer fibers 702 and containing a multicellular construct 704 is shown in FIG. 19. As shown in FIG. 19, the interconnected network of biopolymer fibers 702 can surround the multicellular construct 704, and may be aligned in the radial and/or the circumferential direction.

While the extracellular matrices disclosed herein and illustrated in FIGS. 9-14 and 19 are shown in use with the culture container 200, a person of ordinary skill in the art will appreciate that the extracellular matrices described above can be used in conjunction with any suitable culture container, particularly including, but not limited to, culture containers 200 and 300. The extracellular matrices forming a pathway among the various reservoirs of the culture containers and allowing a chemical gradient to be formed across the extracellular matrix in the culture container. In this way, the extracellular matrices can be readily adapted to a variety of culture container designs to produce a wide array of ready-to-use extracellular matrix assays.

### E. Methods for Making Extracellular Matrices

Also disclosed herein are example methods for making extracellular matrix assays, such as extracellular matrix assay 600 described above. The methods disclosed herein can employ any of the various examples of the rotary stage 100, the culture containers 200, 300, the electronic control module 400, or any combination thereof.

A solution is prepared containing a biopolymer or biopolymer precursor material. The biopolymer or precursor material can be selected from collagen (such as type-I collagen), fibronectin, Matrigel, elastin, laminin, vitronectin, or combinations thereof suitable for forming a three-dimensional scaffold or lattice of biopolymer fibers. Generally, the biopolymer materials are stored and prepared in a non-crosslinked or unpolymerized state. Depending on the biopolymer material used, the solution may require preparation and/or storage at a suitable temperature selected to preserve the polymeric material prior to its use, such as a temperature of 4° C.

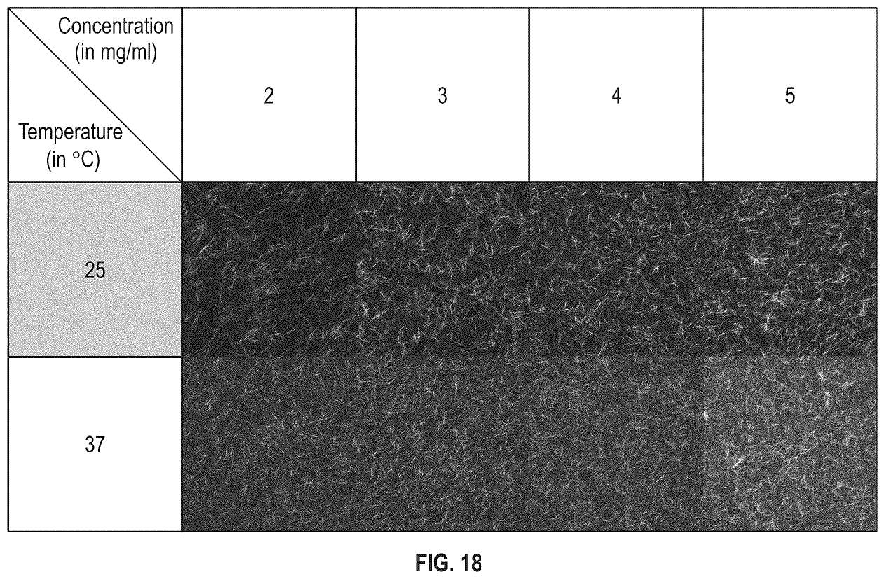

In some examples where an extracellular matrix is being prepared for use with a cell culture, before a cell suspension is added, the biopolymer precursor solution comprises from 1 mg/mL to 50 mg/mL of the biopolymer (i.e., the collagen, the fibronectin, or the Matrigel). More typically, the biopolymer precursor solution can comprise 5-25 mg/mL of the biopolymer. In specific examples, the biopolymer precursor solution can comprise 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL. The concentration of the precursor solution can, in certain examples, be adjusted to control the density of the resulting matrix. For example, as shown in FIG. 18, as the concentration of the precursor solution increases from 2 mg/mL to 5 mg/mL, the density of the resulting matrix can also increase, with more tightly packed fibers and a greater degree of interconnection between the fibers.

Additional compounds, such as cross-linking or gelation agents, or compounds that stimulate particular cell responses may additionally be added, depending on the extracellular matrix assay being prepared. Such compounds can include, for example, Matrigel, fibronectin, arginylglycylaspartic acid (“RGD”), and growth factors such as TGF-β. Cross-linking agents can be selected and included in varying concentrations in order to produce an extracellular matrix with desired mechanical properties, such as strength and porosity of the extracellular matrix. In some examples, the cross-linking agents can be omitted from the extracellular matrix solution. In most cases, the cross-linking agent can be present in an amount ranging from greater than 0 to 10 times the molar concentration of biopolymer molecules.

In some examples, a cellular culture, such as cancer cells (for instance, human breast cancer cell MDA-MB-231), fibroblasts, myofibroblasts, endothelial cells, epithelial cells, immune cells (for instance, T-cells, natural killer cells, and macrophages), stem cells, or neuron cells can be added to the non-crosslinked solution. In some examples, it may be particularly advantageous to add the cellular culture to the extracellular matrix solution before cross-linking and/or gelation of the matrix begins to occur. In this way, when the extracellular matrix is formed from the solution, the cellular culture will be entrained in the network of interconnected biopolymer fibers. However, in other examples, the cell culture may be added after the extracellular matrix has been formed from the solution from gelation and/or cross-linking, such that the cell culture can be absorbed into the network of interconnected biopolymer fibers.

The addition of the cell culture to the biopolymer precursor solution can, in some examples, dilute the biopolymer precursor solution to a concentration of less than 5 mg/mL, such as 0.1-5 mg/mL or more typically 1-3 mg/mL.

Additionally, the biopolymer precursor solution can be neutralized with the addition of various compounds, such as sodium hydroxide (NaOH), to reach a desirable solution pH. The desired pH of any solution will depend on the desired cellular culture to be suspended within the biopolymer precursor solution, or subsequently added to the extracellular matrix. In some examples, the pH can be a substantially physiological pH. In other examples, the pH can have a range such as 6.5-8.0 pH. More typically, the target pH can range from 7.0 pH and 7.5 pH or can range from 7.35 pH to 7.45 pH. In one particular example, the pH can be 7.4. Moreover, various acids, bases, and buffer compounds can be added to the biopolymer precursor solution to reach the desired pH, as will be understood by a person of ordinary skill in the art.

In some examples, the culture container 200, 300, can be treated or prepared to receive the precursor biopolymer solution. In one example, the culture container 200, 300 be treated with a bifunctional agent, such as sulfosuccinimidyl 6-(4′-azido-2′-nitrophenylamino)hexanoate (Sulfo-SANPAH), shown below.

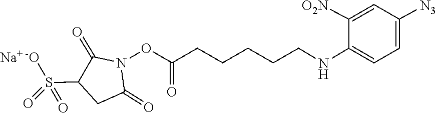

The treated culture container 200 300 can then be exposed to UV light to functionalize the inner surfaces of the culture container 200, 300. After treating and/or preparation, the culture container 200, 300 can be washed to remove any unwanted reactants prior to adding the precursor solution.

When the precursor solution has been prepared with the desired combination of biopolymer materials, cross-linking agents, gelling agents, and/or cell cultures, and the culture container 200, 300, has been adequately prepared to receive the biopolymer precursor solution, the biopolymer precursor solution is then introduced to the culture container to produce an extracellular matrix assay. Extracellular matrix assays can be prepared either with or without agitation.

In some examples, the agitation of the precursor solution in the culture container 200, 300, can be performed using the rotary stage 100. In such examples, the culture container 200, 300 can be positioned beneath the blade assembly 138, such as on the assay holder 172, with the aperture 210, 310 aligned to receive the blade head 152 of the blade assembly 138.

The blade assembly 138 can then be lowered (for example, by actuating the positioning arm 106 to lower the motorized mount 104) in order to insert the blade head 152 into the aperture 210, 310 and immerse the blade head 152 in the precursor solution. In some examples, the blade assembly 138 blade head 152 may be lowered until the blade head 152 is a given distance from the bottom of the aperture 210, 310. When the spacing between the blade head 152 and the bottom of the aperture 210, 310 is small, the bottom surface of the aperture 210, 310 creates a static boundary that provides additional guidance to the self-assembly of biopolymer matrices. Such configuration may also be preferable when culturing large cells which tend to sink to the bottom of the culture matrix. In other examples, such as when culturing small cells with a lower tendency to sink in the solution, it may be convenient to have a larger spacing. When the spacing between the blade head 152 and the bottom of the aperture 210, 310 becomes too low, the flow induced by the blade rotation quickly dissipates; therefore, the range of aligned biopolymer matrix is reduced. In specific examples, the desired spacing between the blade head 152 and the bottom of the aperture 210, 310 can be between 0.02 mm and 5 mm, or more typically, between 0.1 mm and 2 mm.

When the rotary stage 100 includes an elevation detector, such as the capacitance-based elevation detector 190 described above and illustrated in FIG. 4, the elevation detector can be used to control the spacing between the blade head 152 and the bottom of the aperture 210, 310 with a greater degree of precision. For example, a capacitance-based elevation detector 190 can be used to detect the relative spacing between two components, such as an aligner 122, 124, and the assay holder 172. As the aligner 122, 124, and the assay holder 172 draw closer together or move further apart, a measurable change in the capacitance between the components occurs. This allows the spacing between the aligner 122, 124, and the assay holder 172 to be determined at any given point. Because the relative position of the aligner 122, 124, and the blade head is known, and the relative position of the culture container 200, 300, and the assay holder 172 are also known, the distance between the blade head 152 and the bottom of the culture container 200, 300 can be readily calculated, for example by the electronic control module 400 previously described. This allows for precise control over the spacing between the blade head 152 and the bottom of the culture container 200, 300.

The blade assembly 138 can then be rotated such that the blade head 152 rotates within the aperture 210, 310 to create a rotational flow of the biopolymer solution within the culture container 200, 300. The blade assembly 138 can rotate at speeds ranging from greater than 0 RPM to 200 RPM, or more typically from at least 20 RPM to 150 RPM, or at least 50 RPM to 150 RPM. In specific examples, the blade assembly can rotate at 64 RPM or 128 RPM. In alternative examples, where little circumferential alignment of the extracellular matrix fibers is desired, the blade assembly can rotate at speeds ranging from greater than 0 RPM to less than 20 RPM. The rotational motion of the blade drives the flow of the biopolymer solution in a circumferential direction and encourages the nucleation of biopolymer fibers with a circumferential orientation.

Controlling the agitation/stirring parameters, including the rotational speed of the blade assembly 138, the spacing between the blade head 152 and the bottom of the culture container 200, 300, and the agitation time, can provide control over various aspects of the microstructure of the resulting extracellular matrix 500, including the orientation of the biopolymer fibers 502, the degree of cross-linking between the bio-polymer fibers 502, and the coherence between the biopolymer fibers 502.

For example, the rotational speed of the blade can be adjusted to control the orientation of the biopolymer fibers 502 in the resulting extracellular matrix 500. At lower rotational speeds, such as a rotational speed of less than 20 RPM, the biopolymer fibers of the resulting extracellular matrix may be primarily radially oriented (that is, extending from a central location in the observation chamber 206, such as the aperture 210 to an outer diameter of the observation chamber 206). At higher rotational speeds, such as greater than 20 RPM, or more generally 20-150 RPM or 50-150 RPM, or in specific examples, 64 or 128 RPM, the biopolymer fibers of the resulting extracellular matrix may be more circumferentially oriented, that is, tangential to the radial direction. Additionally, the duration of the agitation can be increased or decreased to change the orientation of the biopolymer fibers 502 in the resulting extracellular matrix 500.

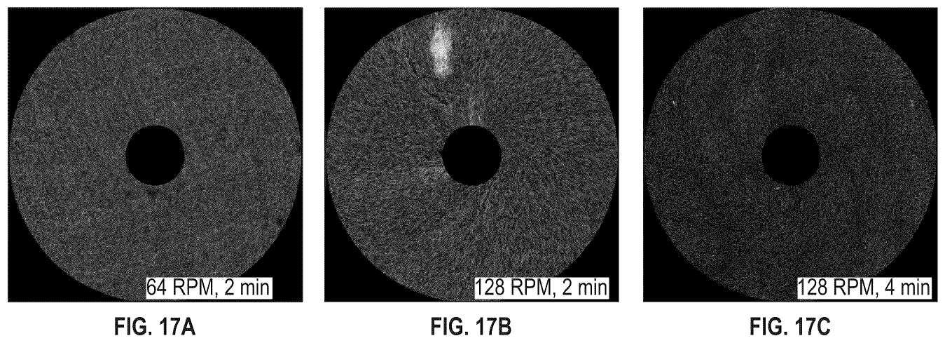

FIGS. 17A-17C show extracellular matrices 500 prepared with agitation for varying times and at varying speeds are shown. FIG. 17A shows an extracellular matrix 500 prepared by rotating the blade assembly 138 at 64 RPM for 2 minutes. The extracellular matrix 500 of FIG. 17A has fibers 502 that extend primarily in the radial orientation, with minimal tangential deviation (i.e., circumferential extension). When the rotational speed is increased from 64 RPM to 128 RPM for 2 minutes, as shown in FIG. 17B, the fibers 502 of the extracellular matrix 500 show a slight tangential deviation from the radial orientation (i.e., a slight circumferential extension). When the agitation time is increased from 2 minutes to 4 minutes at 128 RPM, as shown in FIG. 17C, the fibers 502 of the extracellular matrix 500 exhibit a more pronounced response, and a principally circumferential orientation. As shown in FIGS. 17A-17C, the circumferential alignment of the extracellular matrices 500 tends to increase with the rotational speed of the blade 152, however because the gelation of the matrix is a continuous process, and the alignment of the fibers of the extracellular matrix 500 is affected by the boundary conditions of the cell culture container 200, 300, the correlation may be non-linear.

In some examples, the polymerization temperature of the extracellular matrix also influences the mechanical properties of the extracellular matrix. For example, as shown in FIG. 18, increasing the polymerization temperature from 25° C. to 37° C., the concentration and density of the interconnected fibers of the extracellular matrix increases, decreasing matrix porosity and increasing stiffness.

Extracellular matrices are typically formed by agitating the extracellular matrix precursor solution in the culture container 200, 300, as described above. In some circumstances, unaligned or radially aligned extracellular matrices, such as that shown in FIGS. 10A and 10B, can be prepared without agitation of the extracellular matrix precursor solution, or with agitation at a low rotational speed (for instance, a rotational speed of the blade assembly 138 of 20 RPM or less).

After the desired agitation of the precursor solution has been completed, the blade assembly 138 can be withdrawn from the aperture 210, 310 and the biopolymer precursor solution. However, in some examples, the blade assembly 138 can be left within the precursor solution to provide an additional nucleation site for the biopolymer fibers.

During the gelation process, the biopolymer can nucleate a plurality of fibers, and the fibers form a network of interconnected fibers that make up the extracellular matrix. The walls of the culture container (such as that provided by the container body 202) and, in some examples, the blade assembly 138, provide a boundary condition and nucleation sites that facilitate the nucleation of biopolymer fibers from the precursor solution.

Over time, biopolymer fibers begin to nucleate from the precursor solution, forming, for example, at the nucleation sites along the container body and/or the blade assembly 138. The orientation of the nucleated fibers can be influenced by the presence or absence of a circumferential flow imparted to the solution by the rotation of the blade assembly 138 as previously described. As the first fibers begin to nucleate from the precursor solution, they generate additional nucleation sites for further fibers, which may tend to have the same or a similar orientation to the first-nucleated fibers. In this way, a network of interconnected and aligned (i.e., radially aligned or circumferentially aligned) fibers can be nucleated from the precursor solution to form an extracellular matrix. The gelation process is allowed to proceed until the extracellular matrix sets, which can take between 5 minutes and 60 hours.

Following the formation of the interconnected network of biopolymer fibers, it may be desirable to incubate any cellular culture included in the extracellular matrix assay. In some examples, this can be accomplished by holding the extracellular matrix assay at a suitable incubation temperature, such as a temperature ranging between 20° C. and 50° C. for a suitable incubation time ranging between 5 minutes and 48 hours.

To encourage growth and multiplication of the cells making up the cellular culture, a growth medium can be added to the assay. The growth medium can vary depending on the cell culture of the assay, and can include Dulbecco's Modified Eagle Medium (DMEM), Minimum Essential Medium (MEM), Roswell Park Memorial Institute (RPMI) 1640 Medium, L15, FBS, CBS, or any combination thereof.

Advantageously, because a wide variety of biopolymers and other compounds may be used to prepare the solution used later to form an extracellular matrix, the method described above can be adapted to a wide variety of extracellular matrix assays, suitable for accommodating a wide variety of cellular cultures under a wide variety of conditions.

In one particular example, a solution of 10 mg/mL type-I collagen is mixed with FITC-conjugated water-soluble type I collagen in an 85% to 15% ratio by volume. The solution can then be pH balanced, for example by adding NaOH or any other suitable acids, bases, or pH balances. The solution is then fixed at 1.5 mg/mL collagen by adding an aqueous cell culture suspension of MDA-MB-231 human breast cancer cells. The MDA-MB-231 cells are then suspended in the collagen solution, and the solution is then neutralized with sodium hydroxide to reach a target pH of 7.4.

The interior of the culture container can be treated with Sulfo-SANPAH and exposed to ultraviolet (UV) light with a wavelength ranging from 320 nm to 350 nm for 120 minutes to functionalize the binding surfaces of the culture container. The culture container can afterward be washed with deionized water, or any other buffer solution, such as phosphate buffered saline, to remove any unwanted compounds.

The prepared biopolymer precursor solution is then added to the functionalized culture container to create an extracellular matrix assay. The biopolymer solution is then agitated using rotary stage 100 described above. A treated blade is positioned in an aperture of the culture container and rotated at 128 RPM for 4 minutes to generate a circumferential flow of the biopolymer precursor solution within the culture container.

The extracellular matrix assay is then kept at room temperature for 25 minutes to allow nucleation and cross-linking of the network of biopolymer fibers that make up the extracellular matrix.

Following the formation of the biopolymer network, the extracellular matrix assay is then incubated at 37° C. for 25 minutes. After the initial incubation period of 25 minutes, a serum-free growth medium is added to the extracellular matrix assay to foster the growth of the MDA-MB-231 cells and/or any other cells, tissues, or cellular constructs in the extracellular matrix.

Also disclosed herein are methods for making extracellular matrices containing multicellular constructs (such as multicellular construct 704 shown in FIG. 19), in lieu of cultures of loose cells. These methods may be similar or substantially identical to those for making extracellular matrices containing loose cells, and may comprise the same or substantially the same steps as those methods, save for the differences outlined herein.

Because a multicellular construct 704 cannot be easily suspended in an extracellular matrix precursor solution, the multicellular construct 704 is typically not mixed with the matrix precursor solution prior to its introduction to the culture container 200, 300. Instead, the multicellular construct 704 can be added to the culture container 200, 300 before, after, or at substantially the same time as a separate precursor solution is added to the culture container 200, 300.

The blade assembly 138 is then introduced to the culture container 200, 300 containing the multicellular construct 704 and the extracellular matrix precursor solution, and lowered towards the multicellular construct 704 until the blade head 152 is positioned above the multicellular construct 704 at a distance ranging from 10 μm to 200 μm. The distance between the blade head 152 and the multicellular construct 704 can be determined optically, or by image contrast.

When the blade head 152 is positioned with the desired vertical spacing from the multicellular construct 704, the extracellular matrix precursor solution can be agitated by rotating the blade head, as described herein in relation to the formation of extracellular matrices containing cultures of loose cells, to form an extracellular matrix 706 comprising a network of interconnected biopolymers 702 around and adjacent to the multicellular construct, as shown in FIG. 19. Because the multicellular construct 704 is generally not large enough to disturb the flow pattern of the precursor solution within the culture container 200, 300, similar or substantially identical rotational velocity and agitation time parameters can be used for the formation of the extracellular matrices in these examples.

### F. Methods for Conducting Assay Studies

Also disclosed herein are methods for conducting mechanical, chemical, and/or mechanochemical assays using the culture containers with embodiments of disclosed textured extracellular matrices.

In a typical example assay study method, an extracellular matrix assay, such as the assay 600 comprising an extracellular matrix 500 and a cell culture container 200, 300, is exposed to one or more chemical cues. For instance, for an extracellular matrix assay including the culture container 200, the second reservoir 214 of the culture container can be filled with a serum-rich solution, the serum comprising one or more compounds that stimulate cell growth and survival, such as FBS and CBS, which may be a generic chemoattractant for many types of cells. The first reservoir 208 can be left free of serum or filled with a serum solution at a lesser concentration.

With the serum-rich solution in the second reservoir 214, and a serum-free or lower concentration serum solution in the first reservoir 208, a chemical gradient is formed between the two reservoirs. Because the extracellular matrix extends between the two reservoirs, the cellular culture or the multicellular construct embedded in the extracellular matrix may respond to the chemical stimulus provided by the chemical gradient.

Over time, the cells of the cellular culture or the multicellular construct may move towards or away from the first or second reservoir, depending on the specific serum used and the concentration differences between the first reservoir and the second reservoir. In the absence of any chemical stimulus, the motion of the individual cells of the culture will be essentially random along the biopolymer fibers of the extracellular matrix. Therefore, any net travel of the cells towards or away from the second reservoir 214 can be attributed to the chemotaxis response of those cells. To quantify the strength of the chemotaxis, in the case that the direction of the chemoattractant gradient is toward the center of the observation chamber, the response of the cells to the chemical gradient, the mean chemotaxis index (C̅I̅) can be expressed as:

C̅I̅=\hat{v}·(−\hat{r})

where \hat{v} is a unit vector along the direction of the cell velocity, and −\hat{r} is the direction of chemotaxis. C̅I̅ ranges between 1 and −1, with greater values indicating more efficient migration behavior towards or away from the source of the chemical stimulus in the second reservoir 214. In some examples, C̅I̅ can be calculated from the net movements of the cells of the cell culture can be tracked over a period of time as desired, such as a time period over which cell migration continues.

In some cases, cell circularity is a more desirable variable to quantify the response of the cells to the gradient of a stimulus, which can be mathematically expressed as:

$$circularity = 4 π \frac{area}{{perimeter}^{2}}$$

where “area” is the area covered by the cell and the “perimeter” is the outer perimeter of the cell, including any protrusions or invadopodia. Circularity will therefore be 1 for a circular cell, and less than 1 as the cell begins to assume an elliptical shape or extends invadopodia and/or other protrusions; this may occur when a cell moves towards or away from a chemical stimulus.

It is also possible to measure the effects of combined mechanical and chemical signals on the morphology and the motility of cells' migration within the extracellular matrix of the extracellular matrix assay. For example, when mechanical and chemical cues, such as contact guidance and chemotaxis, respectively, are parallel or substantially parallel (for example, in the case of a radially aligned extracellular matrix 500 with substantially linear biopolymer fibers extending between the first reservoir 208 and the second reservoir 214), cells are not exposed to competing stimuli. Accordingly, the cells may tend to remain substantially elliptical, defined herein as having 80% or more of the circularity of a perfect ellipse with the same aspect ratio. When mechanical and chemical cues operate in different directions, however, such as is the case for an extracellular matrix assay 600 having an extracellular matrix with circumferentially oriented fibers, the cells may more frequently deviate from a substantially elliptical state in response to competing mechanical and chemical cues.

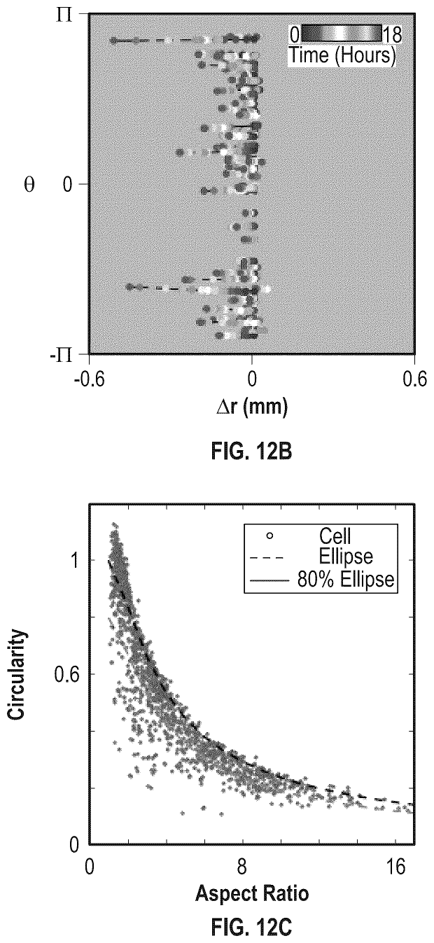

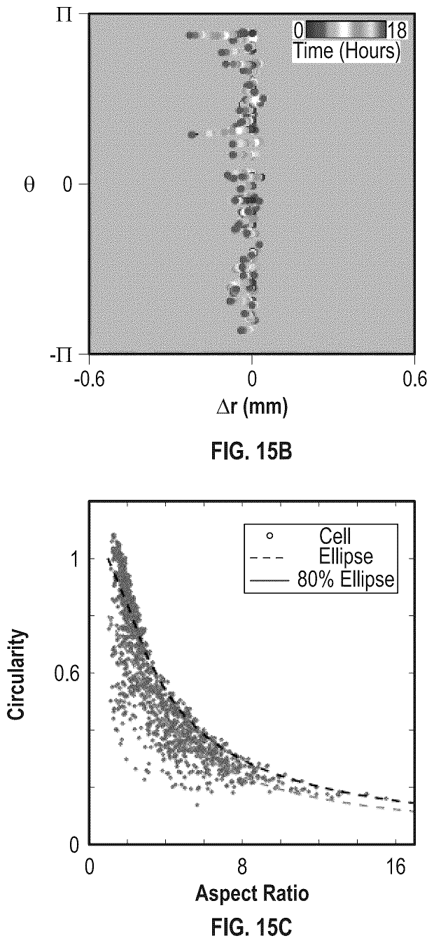

FIGS. 12 and 15 show the response of cells to combined mechanical and chemical signals in both a radially-aligned (FIG. 12) extracellular matrix 500 formed without rotational agitation, or with only low velocity rotational agitation, of the biopolymer precursor solution, and a radially- and circumferentially-aligned (FIG. 15) extracellular matrix 500 formed by rotationally agitating the biopolymer precursor solution. In both examples, the extracellular matrix 500 is added to a culture container 200 to form an extracellular matrix assay 600. The inner reservoir (second reservoir 214) is filled with 20% by volume serum-rich L15 medium, and the outer reservoir (208) is filled with a serum-free L15 medium.

As shown in FIG. 10, when the fibers 502 of the extracellular matrix 500 are radially oriented, the cues from contact guidance and chemotaxis are substantially in parallel. As such, as shown in FIG. 12C, approximately 80% of the cells observed remain substantially elliptical and approximately 20% of the cells are characterized as strongly protrusive. Moreover, because the mechanical and chemical cues are not competing, and instead tend to encourage cellular motion in the same direction, the mean chemotaxis index, C̅I, of cells in the radially oriented extracellular matrix 500 is 0.3, with an average net radial displacement of 85 μm towards the second reservoir 214 over 18 hours.

As shown in FIG. 13, by contrast, when the fibers of the extracellular matrix are circumferentially aligned (i.e., tangentially aligned relative to the direction of the chemical gradient between the first reservoir 208 and the second reservoir 214), the cues from contact guidance and chemotaxis are not in parallel and provide conflicting influence on the cellular response observed. As such, as shown in FIG. 15C, approximately 80% of the cells observed remain substantially elliptical, and approximately 25% of the cells are characterized as strongly protrusive.

Moreover, because the mechanical and chemical cues are divergent and tend to encourage cellular motion in different directions, the mean chemotaxis index, C̅I̅, of cells in the circumferentially-oriented extracellular matrix 500 is 0.14, and the average net radial displacement of the cells is 35 μm towards the second reservoir 214 over 18 hours.

As can be seen from these illustrative examples, the extracellular matrix assays provide the ability to analyze a wide variety of cell cultures and/or multicellular constructs under a variety of chemical and mechanical stimuli and cues. Moreover, because the extracellular matrix assays disclosed herein allow for those mechanical and chemical cues to be measured in parallel, the combined effect of parallel or competing mechanical and chemical cues on the behavior of the cells being studied can also be observed.

Because cells in a living organism are likely to be exposed simultaneously to both mechanical cues and physical cues, a mechanical response (e.g., contact guidance) and a chemical response (e.g., chemotaxis) are likely to occur simultaneously. Thus, the extracellular matrix assays disclosed herein allow for more faithful replication of in vivo conditions, and thus a more helpful analysis of the response of cell cultures to chemical and mechanical cues.

<<Inventors, if there is anything different between the response analysis of multicellular constructs and cell cultures (that is, loose cells), for example, if the multicellular constructs are not expected to demonstrate a chemotaxis response, and instead some other response is measured when testing them,

[symbol]

### IV. EXAMPLES

The following example is provided to illustrate certain features of disclosed embodiments. A person of ordinary skill in the art will appreciate that the scope of the invention is not limited to the particular features described by this example.

### Example 1

To demonstrate the functions of MC3A, the migration and morphology of human breast cancer (MDA-MB-231) cells in a three-dimensional extracellular matrix were studied. The extracellular microenvironment was engineered to simultaneously establish a chemotactic serum gradient and contact guidance in converging or diverging directions of chemotaxis.

### A. Results

The MC3A comprises a rotary stage that is controlled via a computer interface and a disposable culture insert comprising a blade port, as illustrated in FIG. 9. The rotary stage incorporates translation motors to move a spinning head. The spinning head consists of a blade coupled with a rotary motor. When making samples, the motors are manually controlled or follow pre-programmed protocols to dip the blade through the blade port of culture inserts. The spinning of the blade within the culture insert initiates self-assembly (that is, polymerization) of the extracellular matrix. After initiating the extracellular matrix self-assembly, the blade is withdrawn from the culture insert, and the culture insert can thereafter be manipulated or imaged in ways similar to a tissue culture petri dish.

To produce a desirable extracellular matrix microstructure, MC3A takes advantage of a blade with an experimentally optimized tip shape to guide the gelation of an extracellular matrix polymer solution. Additionally, the spinning of the blade creates a flow in the extracellular matrix solution. The flow is both driven by the rotational motion of the blade, as well as constriction walls built inside cell culture insert. After setting the initial flow, the blade exits the solution and the polymer solution is allowed to solidify into a biopolymer network with a microstructure defined and/or influenced by the initial flow. The shape of the blade, its rotation protocol, and the geometry of constriction walls of the culture insert work synergistically to make tissue mimicking biopolymer networks.

A pair of open channel reservoirs are placed above the engineered extracellular matrix microstructure delivery of soluble factors. The reservoirs can be filled with the same or different chemicals to control the biochemical microenvironment of the cells. For example, the inner reservoir can be filled with a growth medium supplemented with a 20% volume concentration of serum, while the outer reservoir is filled with a growth medium without serum. The passive diffusion between the reservoir therefore sets up a serum gradient, which drives the chemotactic motion of MDA-MB-231 breast cancer cells.

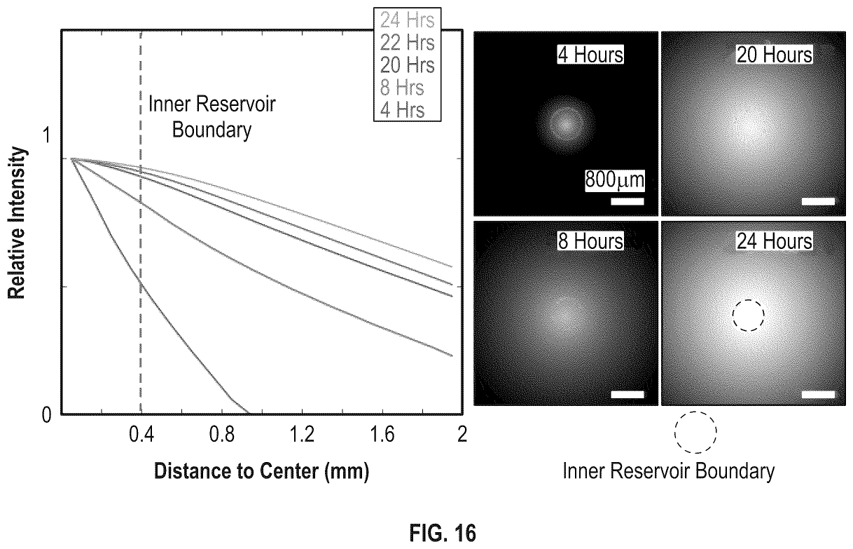

To characterize the microenvironment of cells created by MC3A, the profile of diffusive factors is examined. To this end, the extracellular matrix is constructed with 2 mg/mL type-I collagen matrices. The inner reservoir is then filled with rhodamin B in Phosphate-buffered saline (“PBS”) solution and fill the outer reservoir with pure PBS. The fluorescent intensity of rhodamine B is then measured, which provides a proxy value for the concentration profile of diffusive factors. As shown in FIG. 16, the fluorescent intensity uniformly expands from the inner reservoir in the radial direction towards the outer reservoir. Eight hours after filling the reservoirs, an approximate linear gradient has been established along the radial direction in the extracellular matrix outside of the inner reservoir. The intensity profile continues to stabilize, and after 12 hours of passive diffusion, the intensity gradient in the radial direction reaches a steady state that can last more than 12 hours. In the current example, the inner and outer reservoirs each has a capacity of 1.5 mL. Increasing the dimensions of the reservoirs, such as by raising the height of the retaining walls, can further elongate the duration of the stable chemical gradient.

To characterize the mechanical microenvironment of cells created by MC3A, FITC-labeled type-I collagen is employed such that the extracellular matrix microstructure can be accessed through fluorescent confocal imaging. As representative examples, two distinct configurations produced by executing two simple rotational protocols of MC3A are examined.

When the blade is held still before lifting up from the solidifying collagen solution, the blade combines with the restriction walls in the culture insert to form a boundary condition that facilitates the nucleation of collagen fibers along the radial direction. Confocal images show the expected extracellular matrix microstructure (FIG. 10A). To further quantify the local extracellular matrix geometry, the principal direction (FIG. 10B) and coherence c (FIG. 11) of collagen fiber alignment can be calculated. FIG. 10A shows the spatial distribution of principal fiber direction. Despite the fluctuations expected from the disordered nature of biopolymer networks, collagen fibers evidently show alignment in the radial direction.

In addition to the principal direction, coherence (“c”) measures the level of alignment in the local structure. When all fibers are in parallel, c reaches a maximum value of 1. When the fibers are randomly aligned, c approaches its minimal value of 0. Note that the imaging noise generally suppresses the calculated coherence, and the intrinsic disorder of biopolymer networks forbids perfect alignment. Therefore, the theoretical upper bound of 1 cannot be reached. By comparing calculated coherence with visual inspection and cellular responses, collagen fibers can be considered to be well aligned when c>0.2.

As shown in FIG. 11, the value of coherence starts from around 0.30 near the inner reservoir and gradually decreases to approximately 0.25 at 2 mm away from the device center. The change is well within the range of fluctuations are resulted from extracellular matrix structural disorder. Therefore, the extracellular matrix within the culture insert of MC3A demonstrates a substantially uniform microstructure.

When MDA-MB-231 cancer cells are embedded in the culture insert, the cells experience both the chemotactic serum gradient, and three-dimensional contact guidance from the local fiber alignment. The serum gradient is set by filling the inner reservoir with a 20% volume concentration of serum and filling the outer reservoir with a serum-free growth medium. To characterize the resulted cellular dynamics, the 18 hours after the chemical gradient stabilizes are measured using confocal live cell imaging. FIG. 12A shows a temporal projection of the cells, with each trace representing a single cell from the beginning of the recording (blue, 0 hour) to the end of the recording (red, 18 hours). Because the chemical and mechanical cues are in parallel, cells are shown to move in the radial direction with few diversions.

To further characterize the cell motility, the radial and tangential displacements of a random subset of cells are tracked, as shown in FIG. 12B. Consistent with the temporal projection, cell displacement in the tangential direction (\hat{θ}) is very small. The trajectories in FIG. 12B also reveals that the random walk of a cell often observed in three-dimensional extracellular matrix now occurs with reduced dimension. Cells constantly make 180-degree turns while still showing net displacements towards the center of the device, where serum concentration is higher. Within a frame interval of 1 hour, cells travel at an average instantaneous velocity of 9.5 μm/hr. To quantify the efficiency of cells tracing chemoattractant gradient, we also calculate the mean chemotaxis index C̅I̅:

C̅I̅=\hat{v}·(−\hat{r})

where \hat{v} is the unit vector along the direction of velocity, and −\hat{r} is the unit vector of chemotaxis. C̅I̅ ranges between 1 and −1, with greater values indicating more efficient migration seeking higher chemoattractant concentration. For the cell trajectories shown in FIG. 4B, the mean chemotaxis index is 0.3. The tendency to move along the serum gradient accumulates as cells navigate their microenvironment, such that over 18 hours, the average net radial displacement of cells is 85 μm towards the device center.

In addition to cell motility, cell morphology is also characterized. In particular, the circularity and aspect ratio of binarized single cell images is calculated, as shown in FIG. 12C. Here, circularity is defined as 4πarea/perimeter², which equals 1 for a circle and generally becomes smaller when a cell generates protrusions such as invadopodia.

The present example shows extracellular matrix contact guidance coupled with a chemical cue in parallel to promote cell elongation. About half of the cells sampled in FIG. 12C have an aspect ratio greater than 3, and in over 27% of cases, cells are elongated to have aspect ratios greater than 6.

Despite the elongation, most cells do not deviate from the elliptical shape (as shown by the dashed black line in FIG. 12C) and do not demonstrate significant surface fluctuation. This is believed to be caused by the parallel mechanochemical cues, which provide consistent polarizing signals for cell morphology. To better quantify the morphology of cells, a cell is classified to be strongly protrusive if its circularity is less than 80% of the circularity of an ellipse with the same aspect ratio (indicated in FIG. 12C by the blue dashed line). In the present example, 19% of the observed cells fall into this category. Together, these results show that cell morphology can be characterized as similar to elongated ellipses.

While the parallel chemical and mechanical cues lead to strong cell polarization and dimensional reduction of motility, in physiological conditions (such as in the body of a patient), the two cues may vary independently and motivate cells in different directions. MC3A allows convenient investigation of cellular dynamics in such microenvironment configurations.

As a demonstration, the blade of MC3A is rotated at a constant speed of 120 RPM, which drives the flow of collagen solution and directs the nucleation and growth of collage fibers primarily in the tangential (i.e., circumferential) direction. After 4 minutes of rotation, the blade is lifted from the solidifying collagen solution, and the extracellular matrix self-assembly continues for the next 40 minutes. Confocal images in FIG. 13A and computed local principal direction in FIG. 13B show the expected fiber alignment. Note that because the global flow field resembles a vortex pattern, a radial component of fiber alignment is still observed. In most locations sampled in the device, the angle between the chemical gradient (along radial direction) and contact guidance (extracellular matrix principal direction) is between 45 to 90 degrees (FIG. 14). As a result, cells in this configuration experience chemical and mechanical cues along diverging directions. Here as in the prior case, the strength of the chemical gradient is set by filling the inner reservoir with a 20% volume concentration of serum, and the outer reservoir with a serum-free growth medium.

As the serum gradient drives the MDA-MB-231 cells radially inward, extracellular matrix fibers present contact guidance that steers the cells in the tangential (i.e., the circumferential) direction. The temporal projected confocal recording demonstrates the joined effects of mechanical and chemical cues on cell motility (FIG. 15A). The movement of a random subset of 70 cells is tracked and shown in FIG. 15B. Compared with the previous configuration where collagen fibers align radially, cells in the current configuration exhibit pronounced migration that varies their azimuthal angles. Indeed, instantaneous velocity (approximated by the displacement between 1-hour frame intervals) shows a mean chemotaxis index (C̅I̅) of 0.14, less than half of the value for the radially aligned extracellular matrix. Over the course of 18 hours, the net radial displacement averaged over all tracked cells is 35 μm towards the device center, which is again less than half of the value for the radially aligned extracellular matrix.

The diverging mechanochemical cues also modulate the cell morphology. In particular, only 17% of cells sampled have aspect ratios greater than 6, compared with 27% in the presence of parallel mechanochemical cues. Additionally, of all the cells sampled, 26% show characteristics of strong protrusive cells, compared with 19% in the case of the radially aligned ECM described above.

Together, these results show that when contact guidance and chemotaxis are diverging, cells show a significant reduction in their migration along the chemical gradient. At the same time, cells tend to demonstrate small aspect ratio shapes while featuring strong surface fluctuations.

### B. Conclusion

Many biological processes involve cells navigating a three-dimensional extracellular matrix which contains multiplexed environmental cues. It is conceivable that modeling the cross-talk of biochemical and biophysical factors will improve the physiological relevance of in vitro cell-based assays. Here, a modular platform is presented, which allows the microstructure of a three-dimensional extracellular matrix to be patterned such that contact guidance from the extracellular matrix fiber alignment and spatial gradient of soluble factors can be independently controlled to jointly modulate the cellular dynamics.

Further, MC3A, can generate a sustained chemical gradient over more than 18 hours under passive diffusion. Cells are simultaneously cultured in a thick layer of extracellular matrix (>500 μm) that provides proper three-dimensional support. Additionally, the reservoirs are easily accessible so that additional solutions can be brought in externally to generate a time-dependent chemical environment or to extend the duration of the stable gradient.

The MC3A allows optimization of the geometric design of the boundaries of both spinning blade and dish insert such that the extracellular matrix microstructure can be easily controlled via the programmable rotational protocols of the blade. This approach avoids pre-loaded mechanical stress in the matrix when fiber alignment is induced by external stress. Compared with other flow-based extracellular matrix aligning methods, such as magneto-microfluidics, MC3A produce uniform extracellular matrix alignment over a much larger spatial range. MC3A is also compatible with most tissue-derived proteins (such as collagen, Matrigel, and fibronectin) or synthetic hydrogels. Advantageously, the MC3A can take full advantage of current and future progress in tissue-mimicking biomaterials.

The MC3A features an open channel design, facilitates the delivery of soluble factors to the cells, and the extraction of samples for downstream analysis such as sequencing. This is in contrast to other microfluidics culture and chemotaxis platforms, where the cells in the closed channels are often difficult to be retrieved, especially when they are embedded in solidified matrices.

The MC3A utilizes a modular design such that the shape of the blade, and the geometry of the culture insert can be altered for expanded functionalities. For example, a device to fit two separate sets of extracellular matrices with their inner reservoirs connected, as shown in FIG. 8. With different combinations of media in each reservoir, this culture container can be used to run replicating experiments or to make a side-by-side comparison between distinct microenvironments. Because the culture container can be three dimensionally printed, MC3A allows rapid prototyping to explore expanded functionality.

As illustrated in the above examples, MC3A provides a simple and reliable platform to program complex three-dimensional tissue-mimicking microenvironments. Advantageously, this flexibility of altering the test environment of the culture assay to a variety of test configurations and conditions allows the MC3A to improve techniques for the study of multiplexed chemical and mechanical cues.

### ADDITIONAL EXAMPLES OF THE DISCLOSED TECHNOLOGY

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

Example 1. A device, comprising a cell culture container comprising a container body having a first end portion, a second end portion, and a coverslip attached to the first end portion of the container body, the container body defining a first reservoir, a second reservoir, and an observation chamber positioned between the first reservoir and the coverslip and extending between and open to the first reservoir and the second reservoir;

[symbol]

Example 2. The device of claim 1, further comprising an extracellular matrix positioned within an extracellular matrix chamber and separating the first reservoir from the second reservoir.

Example 3. The device of any example herein, particularly example 2, wherein the extracellular matrix comprises a biopolymer.

Example 4. The device of any example herein, particularly examples 2-3, wherein the extracellular matrix comprises a matrix of interconnected biopolymer fibers.

Example 5. The device of any example herein, particularly example 4, wherein the extracellular matrix comprises collagen, fibronectin, elastin, laminin, vitronectin, a hydrogel, or any combination thereof.

Example 6. The device of any example herein, particularly example 4, wherein the extracellular matrix comprises collagen.

Example 7. The device of any example herein, particularly example 4, wherein the matrix of interconnected biopolymer fibers is radially aligned, and extend from a center portion of the observation chamber towards an outer periphery of the observation chamber.

Example 8. The device of any example herein, particularly example 4, wherein the matrix of interconnected biopolymer fibers is circumferentially aligned, and extend circumferentially within the observation chamber.

Example 9. The device of any example herein, particularly examples 2-8, wherein the extracellular matrix comprises collagen IV, laminin, perlecan, and growth factors.

Example 10. The device of any example herein, particularly examples 2-9, wherein the extracellular matrix comprises collagen, fibronectin, elastin, laminin, vitronectin, poly (ethylene glycol), alginate, gelatin, silk fibroin, polyethylene glycol diacrylate, hyaluronic acid, or any combination thereof.

Example 11. The device of any example herein, particularly examples 2-10, further comprising a cell culture disposed in the extracellular matrix.

Example 12. The device of any example herein, particularly example 11, wherein the cell culture comprises one of cancer cells, endothelial cells, or immune cells.

Example 13. The device of any example herein, particularly example 12, wherein the cell culture comprises MDA-MB-231 cells.

Example 14. The device of any example herein, particularly examples 1-13, wherein the cell culture container further comprises an aperture extending from the observation chamber to the second end portion of the cell culture container and configured to receive a portion of an agitation device and admit a rotatable agitator into the observation chamber.

Example 15. The device of any example herein, particularly example 14, wherein the cell culture container further comprises a channel extending from the aperture to the second reservoir, and the channel, the aperture, and the observation chamber define a fluid pathway from the first reservoir to the second reservoir.

Example 16. The device of any example herein, particularly examples 1-15, wherein the first reservoir includes a first solution and the second reservoir includes a second solution having a different concentration than the first solution, thereby establishing a chemical gradient is formed between the first reservoir and the second reservoir.

Example 17. A method, comprising preparing a precursor solution comprising a biopolymer; adding the precursor solution to a culture container comprising a first reservoir, a second reservoir, and a chamber separating the first reservoir and the second reservoir and forming a fluid pathway from the first reservoir to the second reservoir; retaining the precursor solution in the culture container to nucleate a plurality of biopolymer fibers, and forming an extracellular matrix comprising the plurality of biopolymer fibers in the chamber.

Example 18. The method of any example herein, particularly example 17, further comprising adding a cell culture to the precursor solution before adding the precursor solution to the culture container.

Example 19. The method of any example herein, particularly example 18, further comprising adding a cell culture to the extracellular matrix after the biopolymer fibers have been nucleated.

Example 20. The method of any example herein, particularly examples 18-19, wherein the cell culture comprises one of cancer cells, endothelial cells, or immune cells.

Example 21. The method of any example herein, particularly example 20, wherein the cell culture comprises MDA-MB-231 cells.

Example 22. The method of any example herein, particularly examples 17-21, further comprising adding one or more of an acid, a base, a buffer compound, a gelation agent, a cross-linking agent, or any combination thereof.

Example 23. The method of any example herein, particularly examples 17-22, further comprising agitating the precursor solution within the chamber to create a flow within the chamber.

Example 24. A device prepared according to the method of any example herein, particularly example 17, comprising a culture container having a first reservoir, a second reservoir, and a chamber separating the first reservoir and the second reservoir and forming a fluid pathway from the first reservoir to the second reservoir.

Example 25. The device of example 24, further comprising an extracellular matrix comprising a network of interconnected biopolymer fibers and a cell culture disposed within the network of interconnected biopolymer fibers.

Example 26. A system, comprising a modular assembly with an interchangeable culture assay inserts for adjustable preparation of a cell culture assay, the assembly comprising a rotary stage having a base portion, a motorized mount comprising a rotating shaft, a motor that drives the rotating shaft, and a blade attached to the rotating shaft, and an adjustable positioning arm extending between the motorized mount and the base portion and movable in a first direction to change a relative position of the motorized mount to the base portion; an assay holder positioned below the motorized mount and configured to receive a modular culture insert; and a modular culture insert, comprising a first reservoir, a second reservoir, a chamber separating the first reservoir and the second reservoir, and an aperture opening into the chamber, the aperture configured to receive the blade of the motorized mount and admit the blade into the chamber when the rotary stage is in an operational state, and when the modular culture insert is received by the assay holder.

Example 27. The system of any example herein, particularly example 26, wherein the blade has a shape that conforms to a geometry of the chamber separating the first reservoir and the second reservoir.

Example 28. The system of any example herein, particularly examples 26-27, wherein the blade comprises a wedge-shaped projection extending radially from the rotating shaft.

Example 29. The system of any example herein, particularly examples 26-28, wherein the blade comprises a hydrophobic coating or a hydrophilic coating.

Example 30. The system of any example herein, particularly examples 26-29, wherein the first direction is a vertical direction.

Example 31. The system of any example herein, particularly example 30, wherein the adjustable positioning arm is movable in a second direction orthogonal to the first direction.

Example 32. The system of claim 31, wherein the adjustable positioning arm is movable in a third direction orthogonal to the first direction and the second direction.

Example 33. The system of any example herein, particularly examples 26-32, wherein the assay holder is movable relative to the base portion of the rotary stage.

Example 34. The system of any example herein, particularly example 33, wherein the assay holder is movable in a second direction orthogonal to the first direction.

Example 35. The system of any example herein, particularly example 34, wherein the assay holder is movable in a third direction orthogonal to the first direction and the second direction.

Example 36. The system of any example herein, particularly examples 26-35, wherein the motor, the adjustable positioning arm, and/or the assay holder is computer controlled.

Example 37. A method for simultaneous measurement of a mechanical stimulus and a chemical stimulus on cells, the method comprising preparing a cell culture assay having a first reservoir, a second reservoir, a chamber in fluid communication with the first reservoir and the second reservoir, and an extracellular matrix disposed in the chamber and comprising a biopolymer and a cell culture; adding a first solution with a first concentration of an active chemical to the first reservoir; adding a second solution to the second reservoir to form a chemical gradient between the first reservoir and the second reservoir across the extracellular matrix; and measuring a response of the cell culture to the chemical gradient between the first reservoir and the second reservoir.

Example 38. The method of any example herein, particularly example 36, wherein the second solution contains no active chemical.

Example 39. The method of any example herein, particularly example 37, wherein the second solution has a second concentration of the active chemical, the second concentration being lower than the first concentration.

Example 40. The method of any example herein, particularly examples 36-39, wherein the active chemical is bone morphogenetic proteins, 8CPT-cAMP, fetal bovine serum calf bovine serum, glucose, cAMP, and TGF-β.

Example 41. The method of any example herein, particularly examples 37-40, wherein the first solution or the second solution comprises phosphate-buffered saline.

Example 42. The method of any example herein, particularly examples 37-41, wherein the active chemical is rhodamin B.

Example 43. The method of any example herein, particularly examples 37-42, wherein measuring the response of the cell culture further comprises measuring a movement of cells within the extracellular matrix.

Example 44. The method of any example herein, particularly examples 37-43, wherein the chemical gradient has a first direction and the extracellular matrix comprises a lattice of interconnected fibers, aligned in a second direction different from the first direction, such that the mechanical stimulus and the chemical stimulus act on the cells in different directions.

Example 45. A device, comprising a cell culture container comprising a container body having a first end portion, a second end portion, and a coverslip attached to the first end portion of the container body, an extracellular matrix comprising a network of interconnected fibers; and a cell culture disposed within the extracellular matrix; wherein the container body defines a first reservoir, a second reservoir, and an observation chamber extending between the first reservoir and the second reservoir, and wherein the extracellular matrix is positioned within the observation chamber and separates the first reservoir from the second reservoir.

Example 46. The device of any example herein, particularly example 45, wherein the extracellular matrix comprises collagen, fibronectin, a hydrogel, or a combination thereof.

Example 47. The device of any example herein, particularly examples 45-46, wherein the extracellular matrix comprises collagen IV, laminin, perlecan, and growth factors.

Example 48. The device of any example herein, particularly examples 45-47, wherein the cell culture comprises one of cancer cells, endothelial cells, or immune cells.

Example 49. The device of any example herein, particularly examples 45-48, wherein the cell culture container further comprises an aperture extending from the observation chamber to the second end portion of the cell culture container.

Example 50. A cell culture container comprising a container body having a first end portion, an open second end portion, and a coverslip attached to the first end portion of the container body; a first reservoir positioned within the container body; a second reservoir positioned within the cell culture container and separated from the first reservoir by a wall; an observation chamber positioned between the first reservoir and coverslip, and open to the first reservoir; an aperture extending from the observation chamber to the open second end portion and configured to receive a rotatable blade assembly and admit the rotatable blade assembly into the observation chamber; and a channel extending from the aperture to the second reservoir, wherein the observation chamber, the aperture, and the channel together form a fluid pathway between the first reservoir and the second reservoir.

Example 51. The cell culture container of any example herein, particularly example 50, wherein the observation chamber is configured to receive an extracellular matrix.

Example 52. The cell culture container of any example herein, particularly example 51, wherein the first reservoir is configured to receive a first solution and the second reservoir is configured to receive a second solution, thereby forming a chemical gradient across the extracellular matrix.

Example 53. A cell culture container, comprising a container body having a first end portion, a second end portion, and a coverslip attached to the first end portion of the container body; a first end reservoir and a second end reservoir defined by the cell culture container and open along the second end portion of the container body; a first observation chamber positioned between the first end reservoir and the coverslip, and open to the first end reservoir; a second observation chamber positioned between the second end reservoir and the coverslip, and open to the second end reservoir; an inner wall having a first end portion and a second end portion and disposed within the container body; an inner reservoir defined by the inner wall; a first aperture extending through the inner wall and opening into the first observation chamber; a second aperture extending through the inner wall and opening into the second observation chamber; a first channel extending from the first aperture to the inner reservoir; and a second channel extending from the second aperture to the inner reservoir; wherein the first observation chamber, the first aperture, and the first channel define a fluid pathway between the first end reservoir and the inner reservoir, and the second observation chamber, the second aperture, the second channel define a fluid pathway between the second end reservoir and the inner reservoir.

Example 54. The cell culture container of any example herein, particularly example 53, wherein the first observation chamber and the second observation chamber are configured to receive an extracellular matrix.

Example 55. The cell culture container of any example herein, particularly example 54, wherein the inner reservoir is configured to receive a first solution and the first end reservoir is configured to receive a second solution, thereby forming a chemical gradient across the extracellular matrix between the first end reservoir and the inner reservoir.

Example 56. The cell culture container of any example herein, particularly example 55, wherein the second end reservoir is configured to receive a third solution, thereby forming a chemical gradient across the extracellular matrix between the second end reservoir and the inner reservoir.

Example 57. The cell culture container of any example herein, particularly examples 53-56, wherein the cell culture container further comprises a third end reservoir defined by the container body and in fluid communication with the inner reservoir.

Example 58. The cell culture container of any example herein, particularly example 57, wherein the cell culture container further comprises a fourth end reservoir defined by the container body and in fluid communication with the inner reservoir.

1. A device, comprising:
a cell culture container comprising a container body having a first end portion, a second end portion, and a coverslip attached to the first end portion of the container body, the container body defining a first reservoir, a second reservoir, and an observation chamber positioned between the first reservoir and the coverslip and extending between and open to the first reservoir and the second reservoir.

2. The device of claim 1, further comprising an extracellular matrix positioned within an extracellular matrix chamber and separating the first reservoir from the second reservoir.

3. The device of claim 2, wherein the extracellular matrix comprises a matrix of interconnected biopolymer fibers.

4. The device of claim 3, wherein the extracellular matrix comprises collagen, fibronectin, elastin, laminin, vitronectin, poly (ethylene glycol), alginate, gelatin, silk fibroin, polyethylene glycol diacrylate, hyaluronic acid, or any combination thereof.

5. The device of claim 4, wherein the extracellular matrix further comprises perlecan, and one or more growth factors.

6. The device of claim 3, wherein the interconnected biopolymer fibers are radially aligned and extend from a center portion of the observation chamber towards an outer periphery of the observation chamber.

7. The device of claim 3, wherein the interconnected biopolymer fibers are circumferentially aligned and extend circumferentially within the observation chamber.

8. The device claim 2, further comprising a cell culture disposed in the extracellular matrix, wherein the cell culture comprises at least one of cancer cells, endothelial cells, stem cells, or immune cells.

9. The device of claim 2, further comprising a multicellular structure incorporated in the extracellular matrix, wherein the multicellular structure is one of a tumor spheroid, tissue, organoid, or other multicellular construct.

10. The device of claim 1, wherein the cell culture container further comprises:
an aperture extending from the observation chamber to the second end portion of the cell culture container and configured to receive a portion of an agitation device and admit a rotatable agitator into the observation chamber; and
a channel extending from the aperture to the second reservoir, and the channel, the aperture,
wherein the observation chamber define a fluid pathway from the first reservoir to the second reservoir.

11. The device claim 1, wherein the first reservoir includes a first solution and the second reservoir includes a second solution having a different concentration than the first solution, thereby establishing a chemical gradient is formed between the first reservoir and the second reservoir.

12. A system, comprising:
a modular assembly for use with an interchangeable culture assay insert for adjustable preparation of a cell culture assay, the assembly comprising:
a rotary stage having a base portion, a motorized mount comprising a rotating shaft, a motor that drives the rotating shaft, and a blade attached to the rotating shaft, and an adjustable positioning arm extending between the motorized mount and the base portion and movable in a first direction to change a relative position of the motorized mount to the base portion;
an assay holder positioned below the motorized mount and configured to receive a modular culture insert; and
a modular culture insert, comprising a first reservoir, a second reservoir, a chamber separating the first reservoir and the second reservoir, and an aperture opening into the chamber, the aperture configured to receive the blade of the motorized mount and admit the blade into the chamber when the rotary stage is in an operational state, and when the modular culture insert is received by the assay holder.

13. The system of claim 12, wherein the blade has a shape that conforms to a geometry of the chamber separating the first reservoir and the second reservoir and comprises a wedge-shaped projection extending radially from the rotating shaft.

14. The system of claim 12, wherein the adjustable positioning arm is movable in a second direction orthogonal to the first direction.

15. The system of claim 14, wherein the adjustable positioning arm is movable in a third direction orthogonal to the first direction and the second direction.

16. The system of claim 12, wherein the assay holder is movable relative to the base portion of the rotary stage.

17. A cell culture container, comprising:
a container body having a first end portion, a second end portion, and a coverslip attached to the first end portion of the container body;
a first end reservoir and a second end reservoir defined by the cell culture container and open along the second end portion of the container body;
a first observation chamber positioned between the first end reservoir and the coverslip, and open to the first end reservoir;
a second observation chamber positioned between the second end reservoir and the coverslip, and open to the second end reservoir;
an inner wall having a first end portion and a second end portion and disposed within the container body;
an inner reservoir defined by the inner wall;
a first aperture extending through the inner wall and opening into the first observation chamber;
a second aperture extending through the inner wall and opening into the second observation chamber;
a first channel extending from the first aperture to the inner reservoir; and
a second channel extending from the second aperture to the inner reservoir;
wherein the first observation chamber, the first aperture, and the first channel define a fluid pathway between the first end reservoir and the inner reservoir, and the second observation chamber, the second aperture, the second channel define a fluid pathway between the second end reservoir and the inner reservoir.

18. The cell culture container of claim 17, wherein the first observation chamber and the second observation chamber are configured to receive an extracellular matrix and wherein the inner reservoir is configured to receive a first solution and the first end reservoir is configured to receive a second solution, thereby forming a chemical gradient across the extracellular matrix between the first end reservoir and the inner reservoir.

19. The cell culture container of claim 18, wherein the second end reservoir is configured to receive a third solution, thereby forming a chemical gradient across the extracellular matrix between the second end reservoir and the inner reservoir.

20. A method for simultaneous measurement of a mechanical stimulus and a chemical stimulus on cells using the device of claim 1, the method comprising:
preparing a cell culture assay by forming an extracellular matrix comprising a biopolymer and one of a cell culture or a multicellular construct to the observation chamber;
adding a first solution with a first concentration of an active chemical to the first reservoir;
adding a second solution to the second reservoir to form a chemical gradient between the first reservoir and the second reservoir across the extracellular matrix; and
measuring a response of the cell culture to the chemical gradient between the first reservoir and the second reservoir.
